(19) United States
(12) Patent Application Publication
(10) Pub. No.: US 2026/0014343 A1
(43) Pub. Date: Jan. 15, 2026

(54) PNEUMATICALLY CONTROLLABLE VENT

(71) Applicant: ResMed Pty Ltd, Bella Vista (AU)

(72) Inventors: Luke Andrew STANISLAS, Sydney (AU); Stewart Joseph WAGNER, Hawkesbury (AU); Saxon Alexander BROWN, Sydney (AU); Muditha Pradeep DANTANARAYANA, Sydney (AU)

(21) Appl. No.: 19/257,661

(22) Filed: Jul. 2, 2025

Related U.S. Application Data
(60) Provisional application No. 63/669,534, filed on Jul. 10, 2024.

(51) Int. Cl. A61M 16/20; A61M 16/00; A61M 16/06; A61M 39/00; A61M 39/22

(52) U.S. Cl. CPC A61M 16/201; A61M 16/0003; A61M 16/0616; A61M 16/0683; A61M 39/227; A61M 2039/0009; A61M 2202/0007; A61M 2202/0092; A61M 2205/103; A61M 2205/3344

(57) ABSTRACT

A pneumatically controlled gas washout vent is configured to vent respiratory gas from a patient interface. The gas washout vent includes an inflatable body, a pump configured to pressurize the inflatable body, and housing that enclosed the inflatable body and the pump. An airflow passage extends between the inflatable body and a wall of the housing. The inflatable body is configured to expand upon being inflated by the pump and narrow the airflow passage upon being inflated.

### 1 CROSS-REFERENCE TO RELATED APPLICATIONS

This application claims priority to U.S. Provisional Application No. 63/669,534, filed Jul. 10, 2024 which is hereby incorporated herein by reference in its entirety.

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO₂ to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders:

Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

2.2.3.1.4 Pressurised Air Conduit used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Inc. has developed a number of improved mask vent technologies, e.g. see International Patent Application Publication No. WO 1998/034665; International Patent Application Publication No. WO 2000/078381; U.S. Pat. No. 6,581,594; US Patent Application Publication No. US 2009/0050156; US Patent Application Publication No. 2009/0044808.

Table of noise of prior masks (ISO 17510-2:
2007, 10 cmH2O pressure at 1 m)

| | | A-weighted | A-weighted | |
|---|---|---|---|---|
| | | sound power | sound pressure | |
| | Mask | level dB(A) | dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| Mirage ™ (*) | | | | |
| ResMed | nasal | 36 (3) | 28 (3) | 2000 |
| UltraMirage ™ | | | | |
| ResMed | nasal | 32 (3) | 24 (3) | 2002 |
| Mirage | | | | |
| Activa ™ | | | | |
| ResMed | nasal | 30 (3) | 22 (3) | 2008 |
| Mirage | | | | |
| Micro ™ | | | | |
| ResMed | nasal | 29 (3) | 22 (3) | 2008 |
| Mirage ™ | | | | |
| SoftGel | | | | |
| ResMed | nasal | 26 (3) | 18 (3) | 2010 |
| Mirage ™ FX | | | | |
| ResMed | nasal | 37 | 29 | 2004 |
| Mirage | pillows | | | |
| Swift ™ (*) | | | | |
| ResMed | nasal | 28 (3) | 20 (3) | 2005 |
| Mirage Swift ™ | pillows | | | |
| II | | | | |
| ResMed | nasal | 25 (3) | 17 (3) | 2008 |
| Mirage Swift ™ | pillows | | | |
| LT | | | | |
| ResMed AirFit | nasal | 21 (3) | 13 (3) | 2014 |
| P10 | pillows | | | |

(* one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH₂O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a gas washout vent including an inflatable body that is configured to be pressurized by a source of gas; and configured to regulate a flow of gas washout through the vent.

Another aspect of one form of the present technology is a pneumatically controlled gas washout vent configured to vent respiratory gas from a patient interface, the gas washout vent comprising: an inflatable body; a pump configured to pressurize the inflatable body; housing that enclosed the inflatable body and the pump; and an airflow passage extending between the inflatable body and a wall of the housing, wherein the inflatable body is configured to expand upon being inflated by the pump and narrow the airflow passage upon being inflated.

A central region of the vent may be pneumatically sealed. The housing may include a cap portion positioned on an exterior of the patient interface, and a base portion positioned inside the patient interface. The cap portion may include one or more outlet openings. The airflow passage may be configured to discharge gas washout through the one or more outlet openings. One or more inlet openings may be positioned around a perimeter of the base portion. The inflatable body may be secured to the base portion.

The gas washout may include an air chamber configured to receive a pneumatic output of the pump and configured to discharge gas to the inflatable body.

The inflatable body may be configured to be in a deflated state when the pump is disabled. The inflatable body may be configured so that when the inflatable body is deflated, a cross-sectional width of the airflow passage is greatest. The inflatable body is configured to be incrementally inflated and deflated. The inflatable body may be configured to be dynamically inflated and deflated. An outer perimeter of the inflatable body may be inclined so that a radius of the inflatable body on one side is greater than a radius of the inflatable body on the other side. An outer perimeter of the inflatable body may have a circumferential bead or flange. The pump may be one of a diaphragm pump, a piezoelectric pump, or a peristaltic pump.

Another aspect of one form of the present technology is a patient interface assembly including a sealing structure; a positioning and stabilizing structure configured to support the sealing structure; and the gas washout vent discussed above.

Another aspect of one form of the present technology is a pneumatically controlled gas washout vent configured to vent respiratory gas from a patient interface, the gas washout vent comprising: an inflatable body; a pneumatic inlet configured to receive pressurized gas; an air chamber configured to receive the pressurized gas from the pneumatic inlet and discharge the pressurized gas to the inflatable body; housing that enclosed the inflatable body and the pump; and an airflow passage extending between the inflatable body and a wall of the housing, wherein the inflatable body is configured to expand upon being inflated by the pressurized gas and narrow the airflow passage upon being inflated.

A central region of the vent may be pneumatically sealed. The housing may include a cap portion positioned on an exterior of the patient interface, and a base portion positioned inside the patient interface. The cap portion may include one or more outlet openings. The airflow passage may be configured to discharge gas washout through the one or more outlet openings. One or more inlet openings may be positioned around a perimeter of the base portion. The inflatable body may be secured to the base portion. The inflatable body may be configured to be in a deflated state when a flow of the pressurized gas to the pneumatic inlet is terminated. The inflatable body may be configured so that when the inflatable body is deflated, a cross-sectional width of the airflow passage is greatest. The inflatable body may be configured to be incrementally inflated and deflated. The inflatable body may be configured to be dynamically inflated and deflated. An outer perimeter of the inflatable body may be inclined so that a radius of the inflatable body on one side is greater than a radius of the inflatable body on the other side. An outer perimeter of the inflatable body may have a circumferential bead or flange.

Another aspect of one form of the present technology is a patient interface assembly comprising: a sealing structure; a positioning and stabilizing structure configured to support the sealing structure; and the gas washout vent discussed above.

Another aspect of one form of the present technology is an RPT system comprising: the patient interface assembly discussed above; an air delivery conduit configured to supply a first flow of pressurized gas to the patient interface assembly; and a pump configured to supply a second flow of pressurized gas to the gas washout vent.

The pump may be of a diaphragm pump, a piezoelectric pump, or a peristaltic pump.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

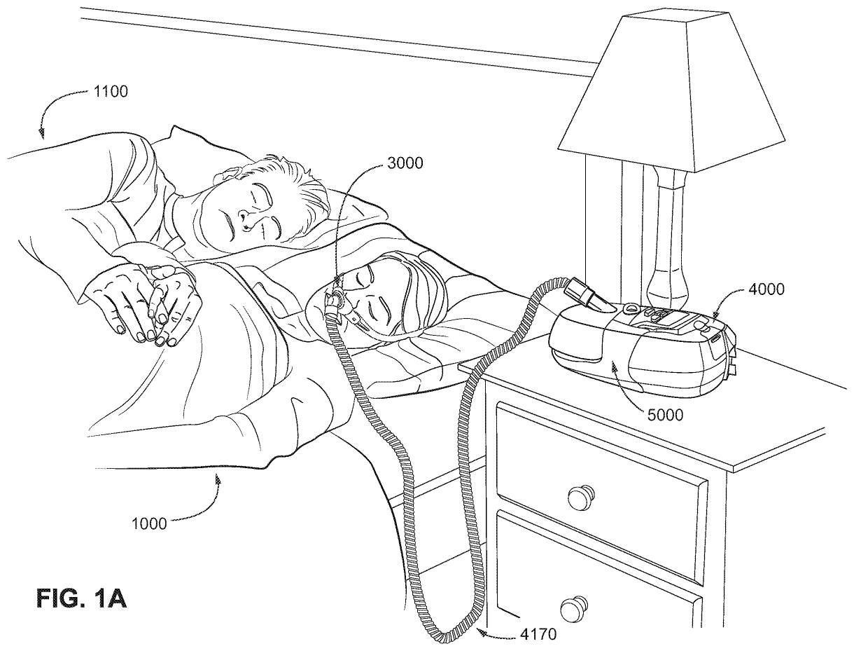

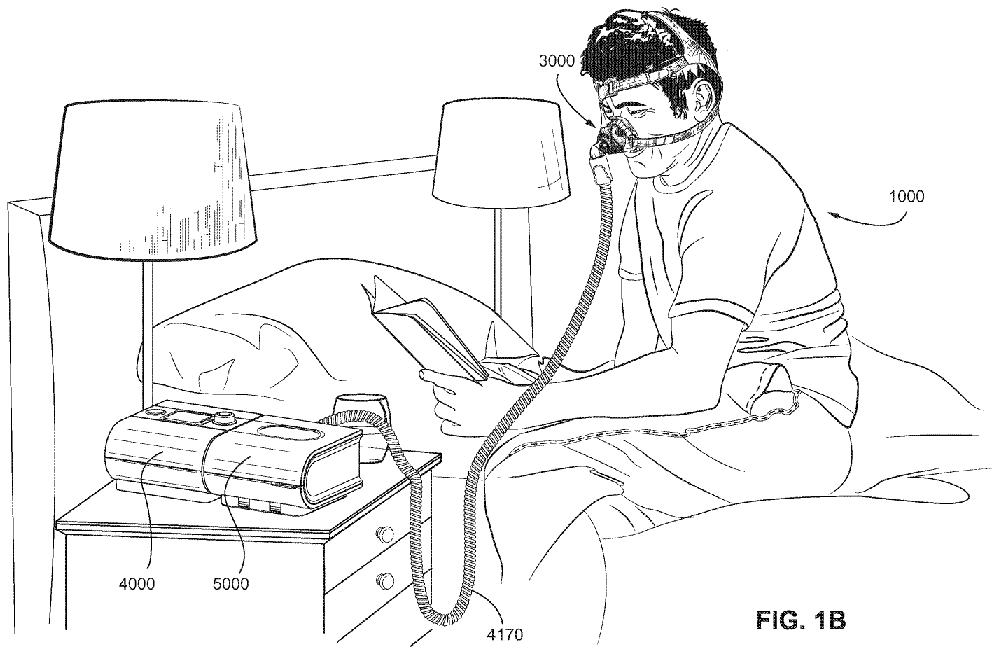

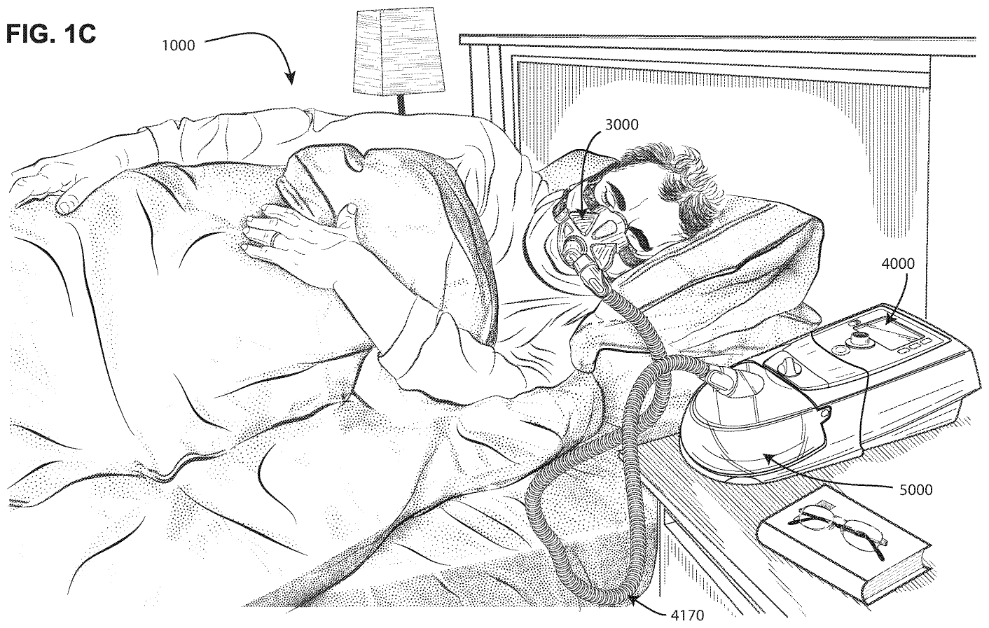

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

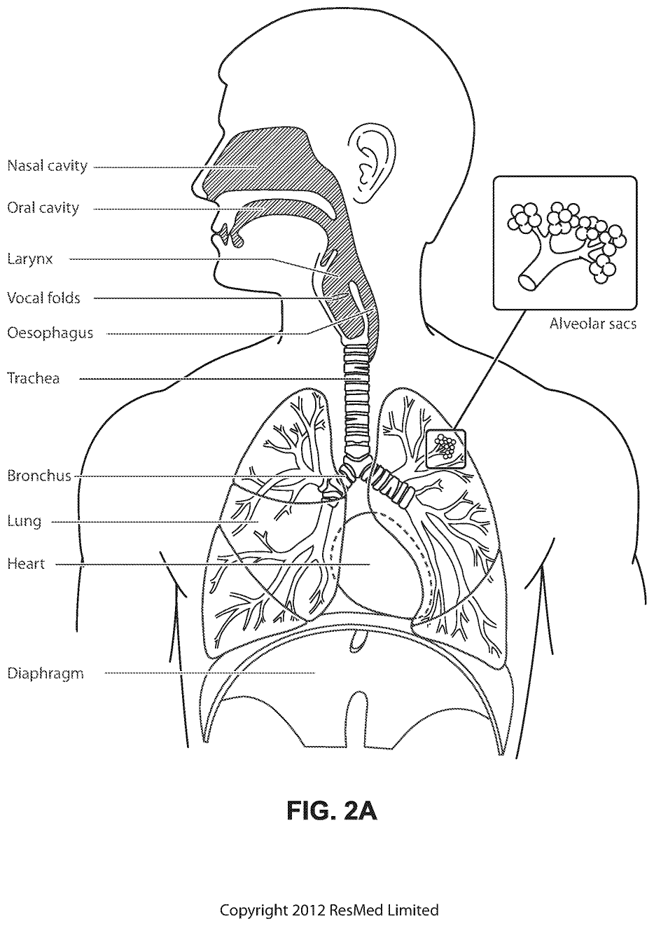

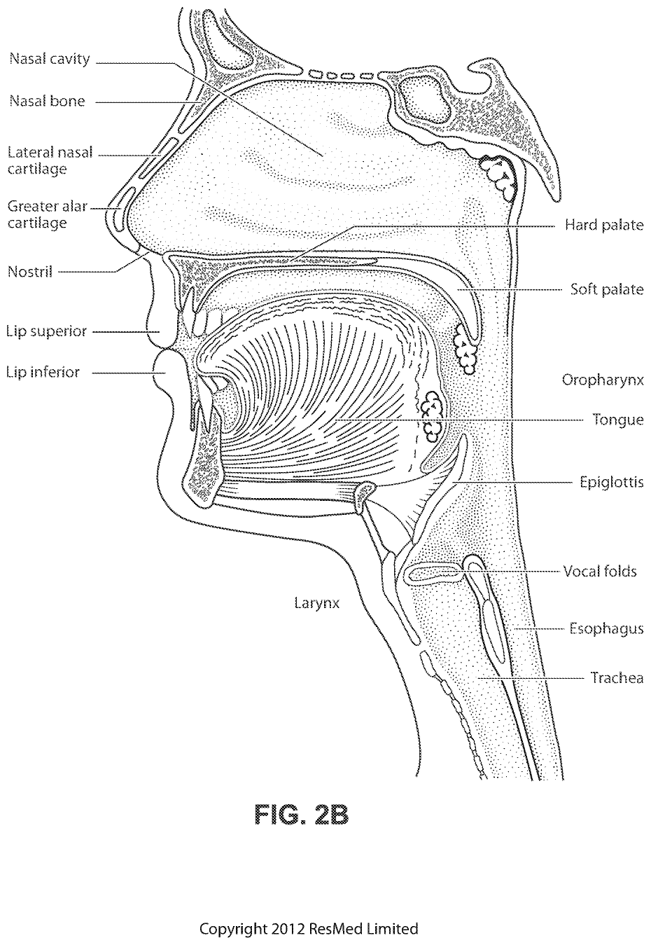

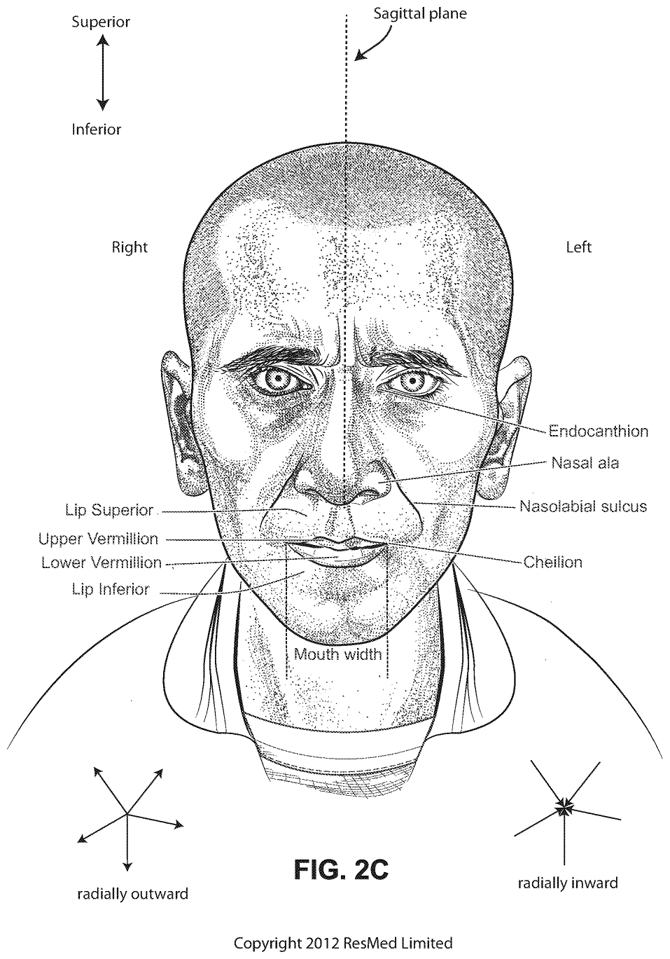

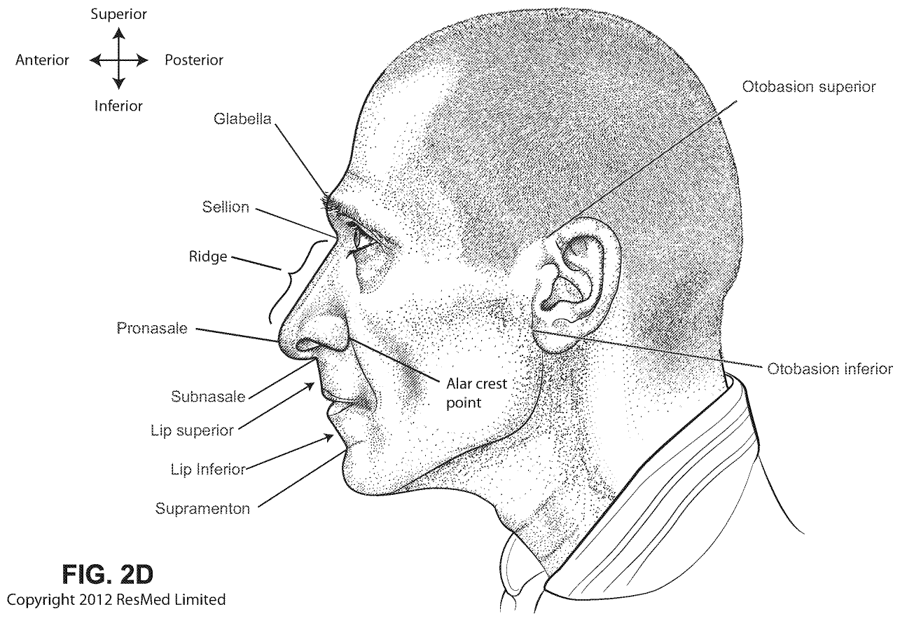

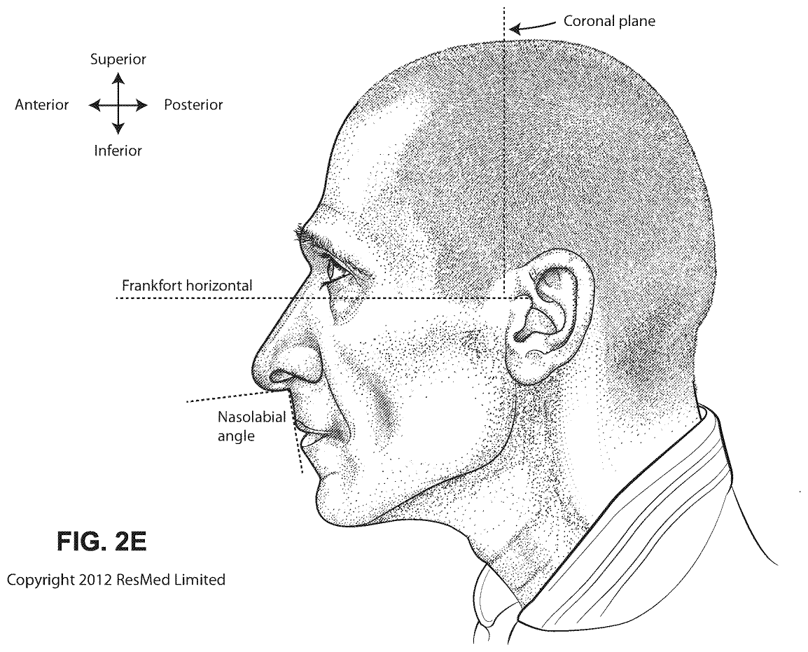

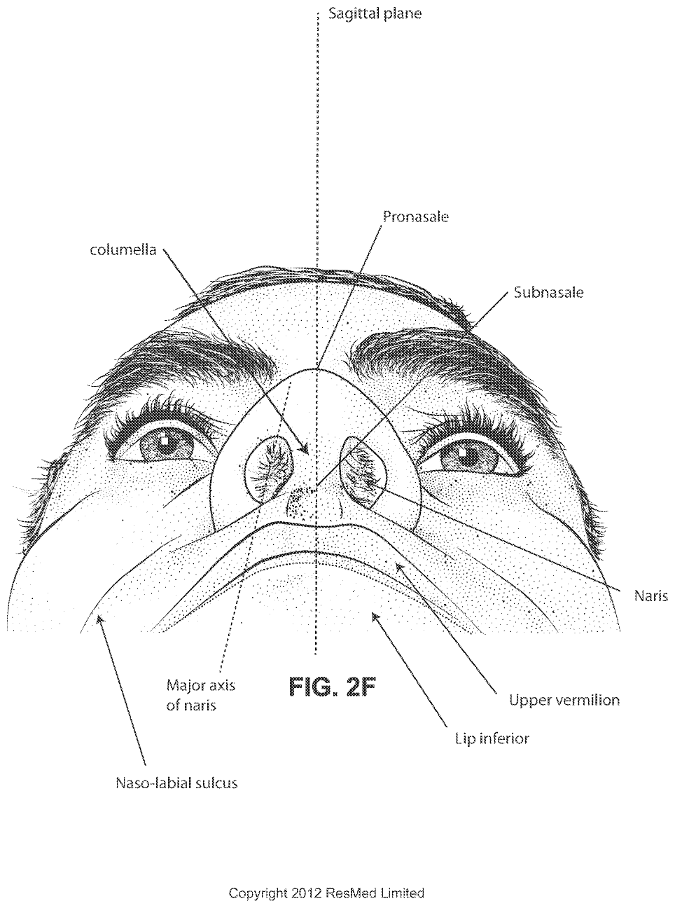

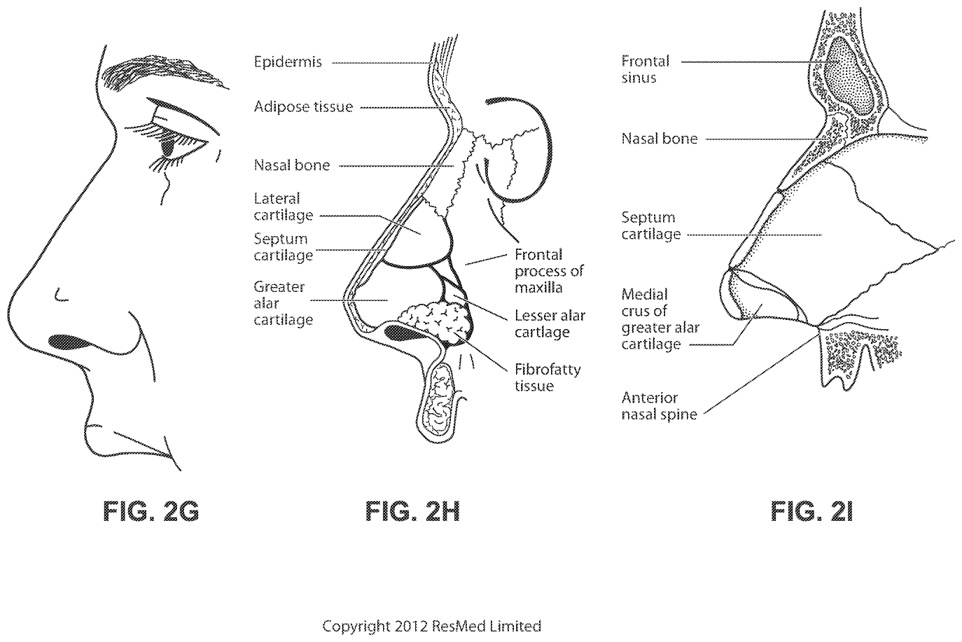

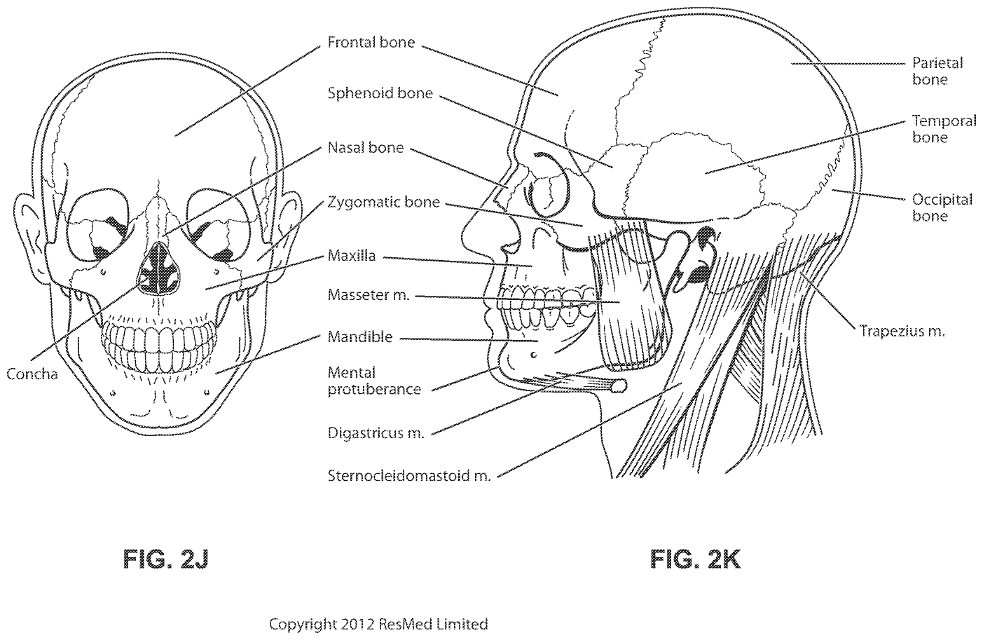

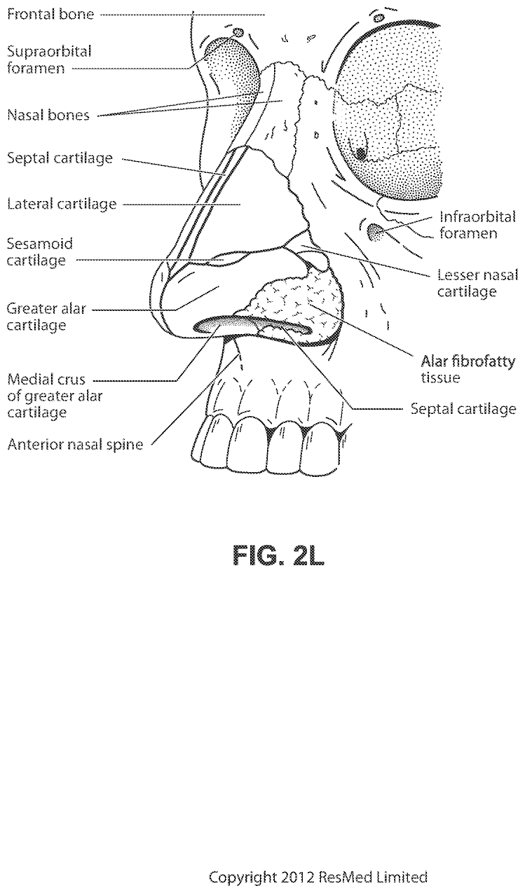

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideopezius.

FIG. 2L shows an anterolateral view of a nose.

### 4.3 Patient Interface

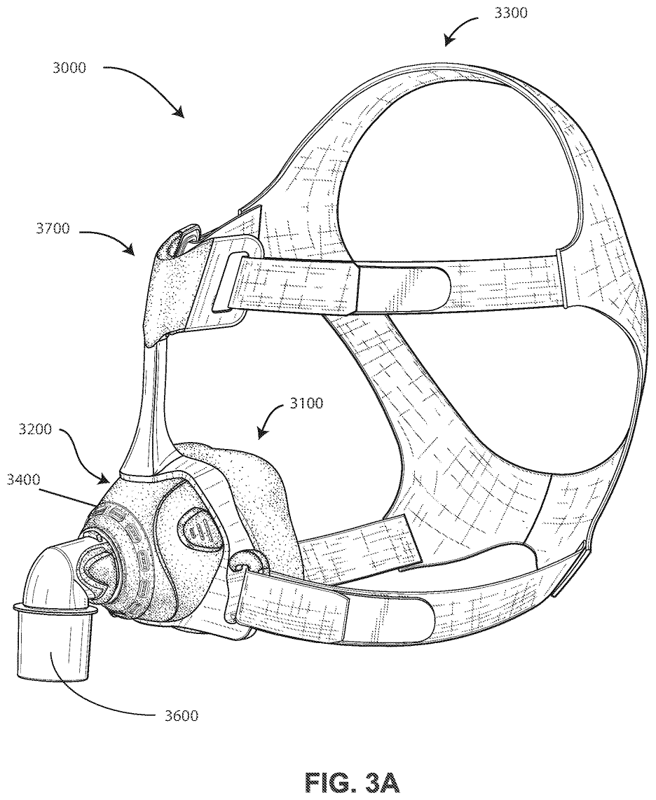

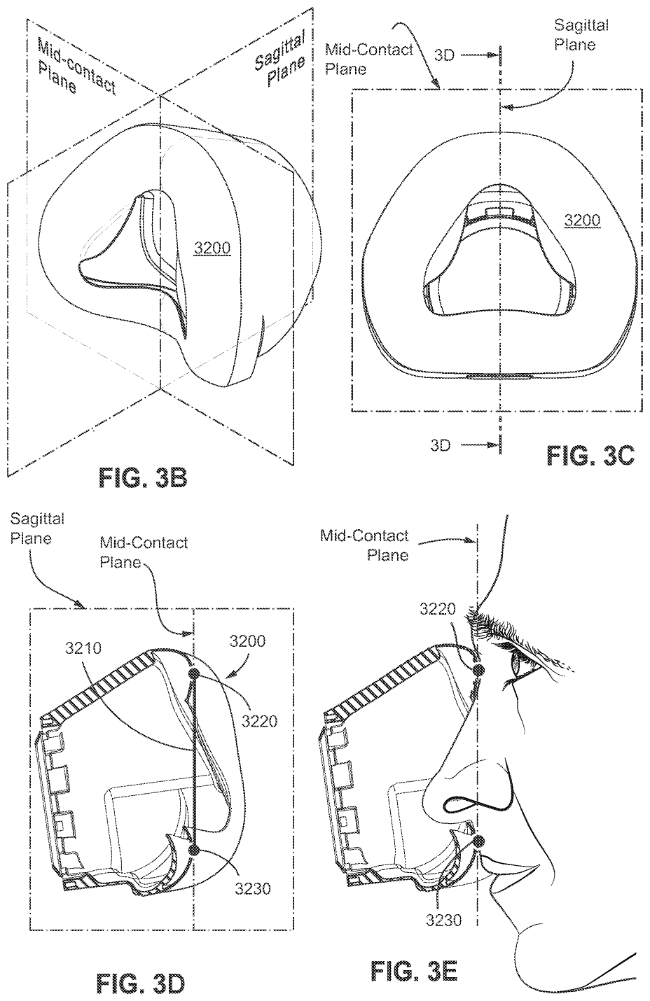

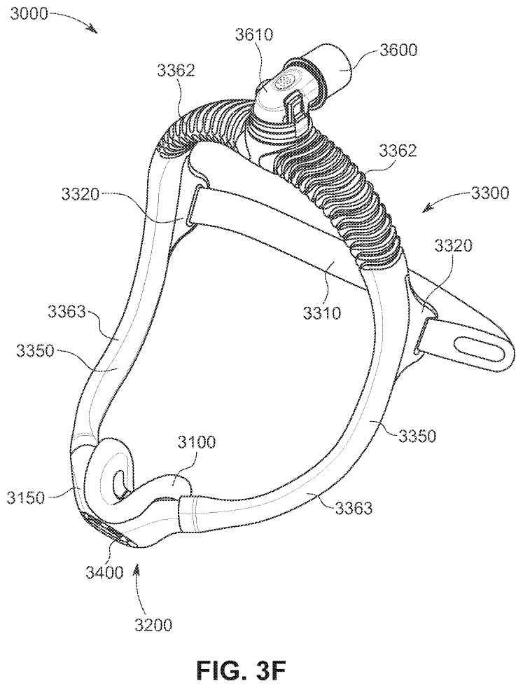

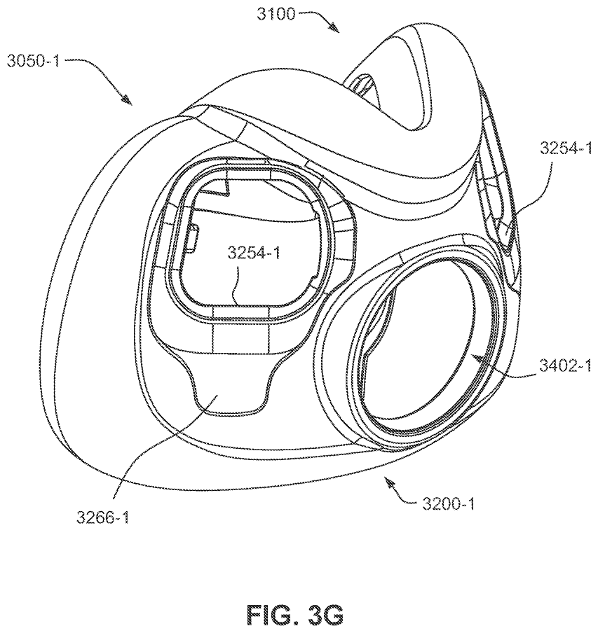

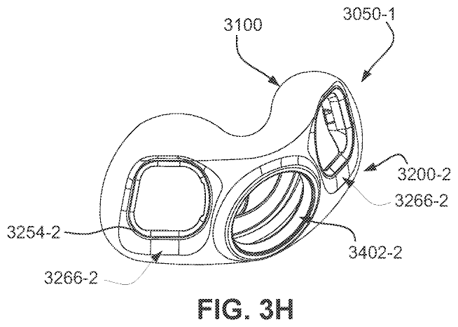

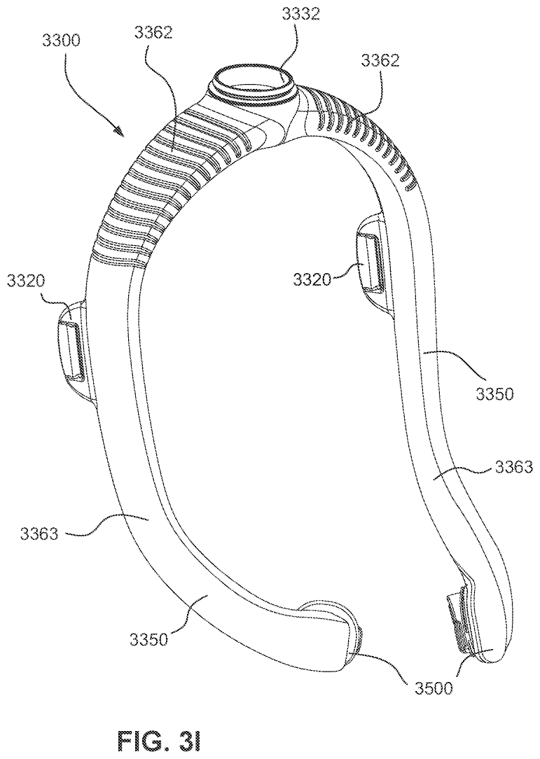

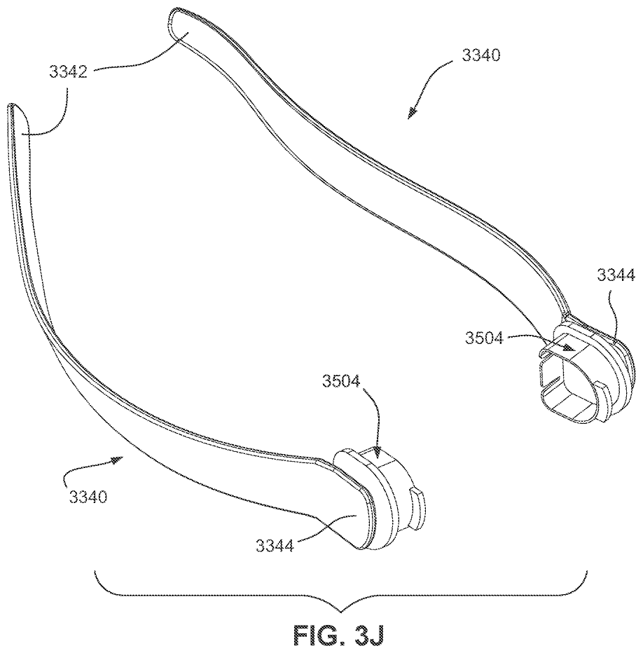

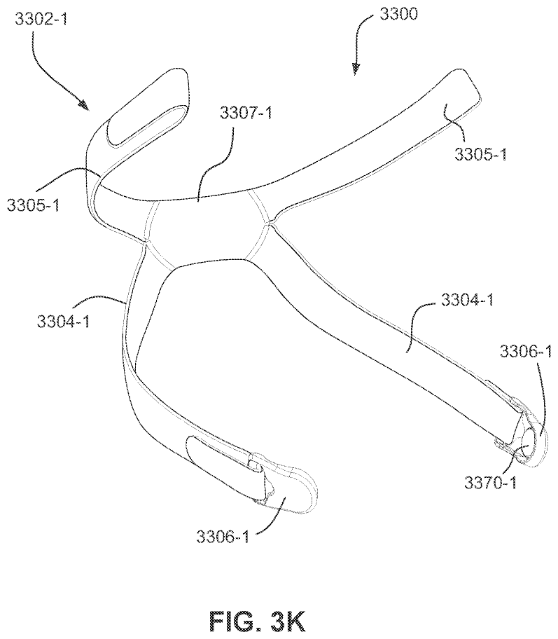

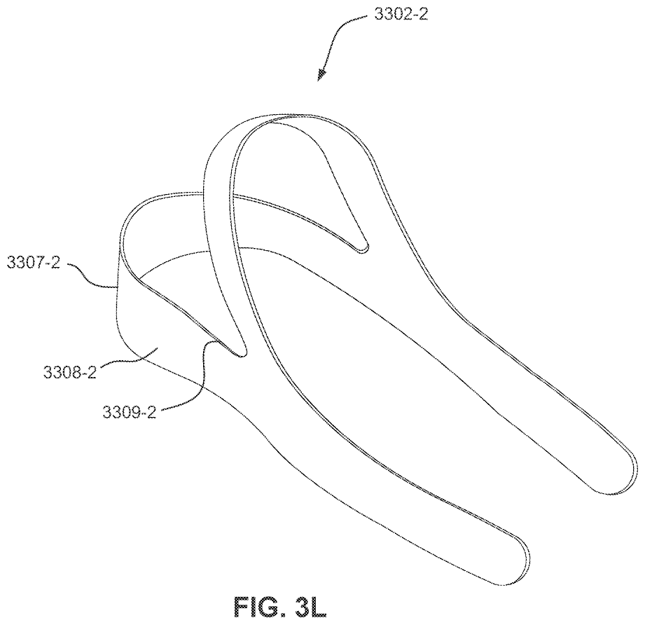

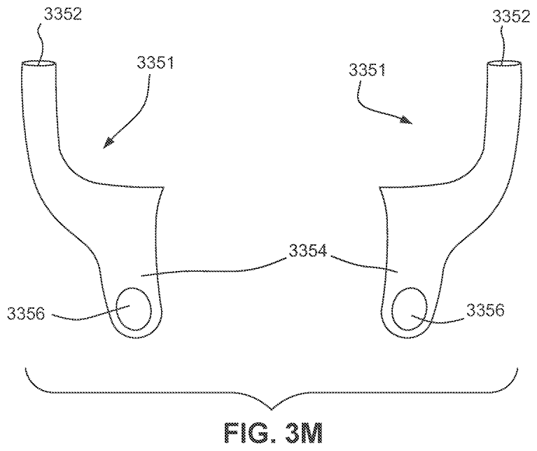

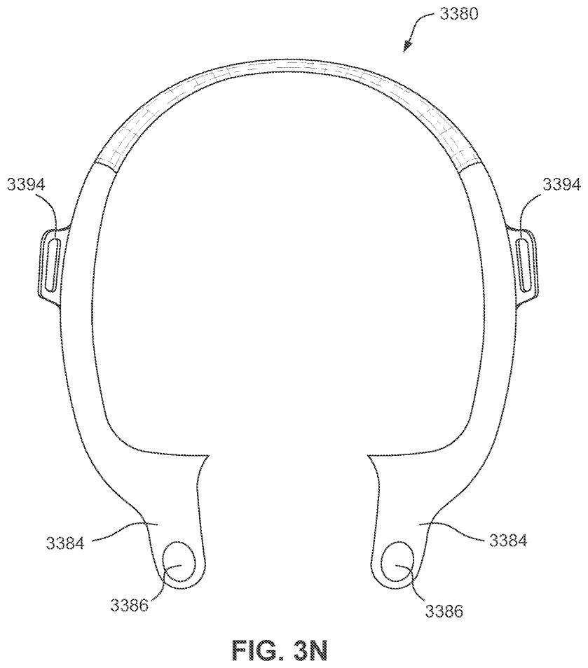

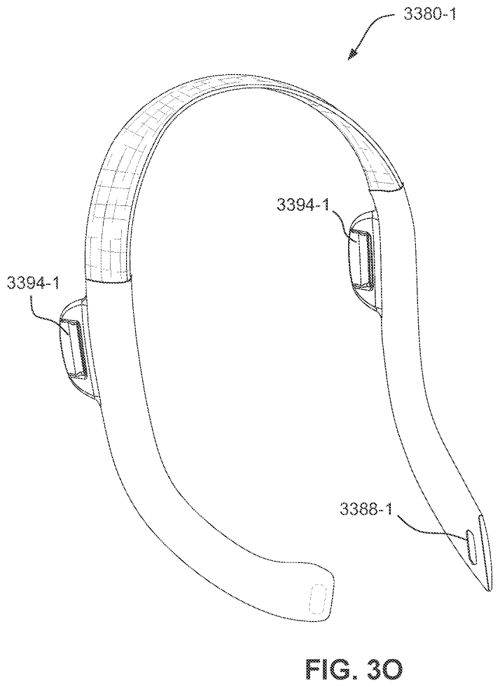

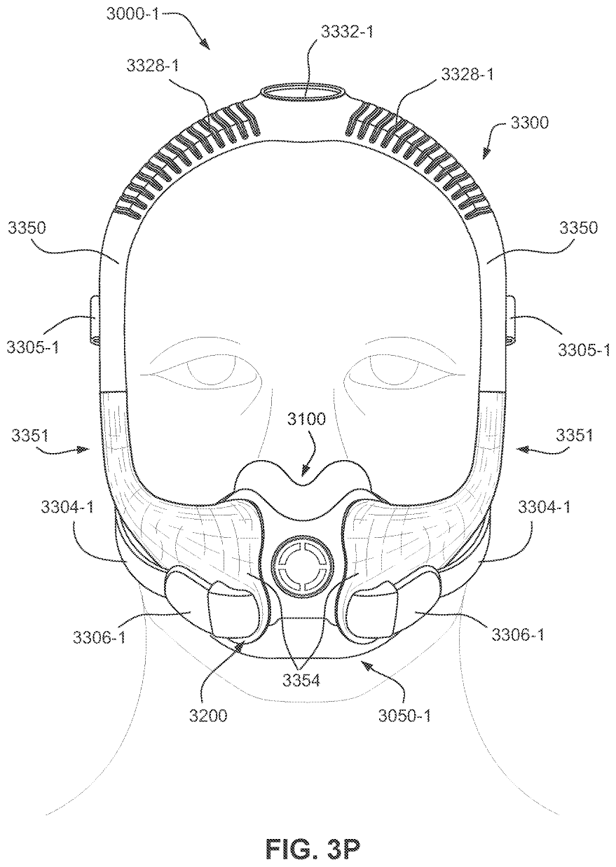

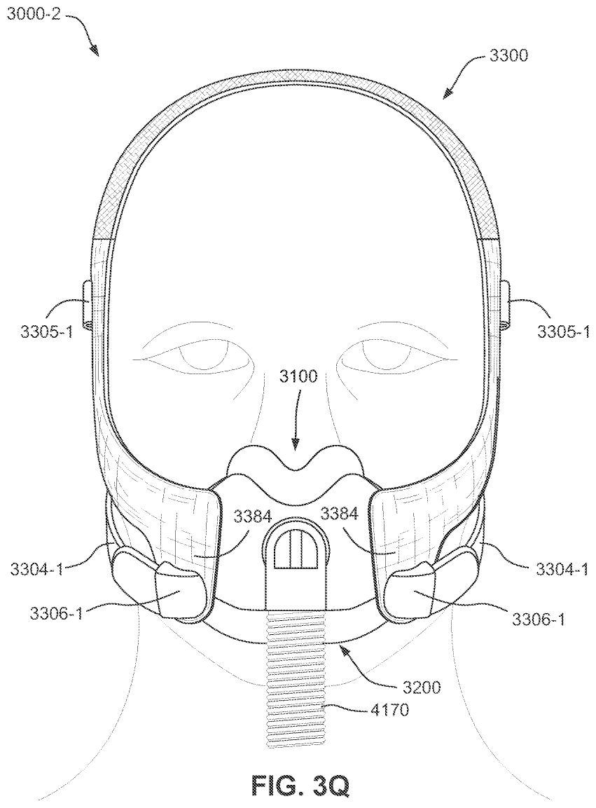

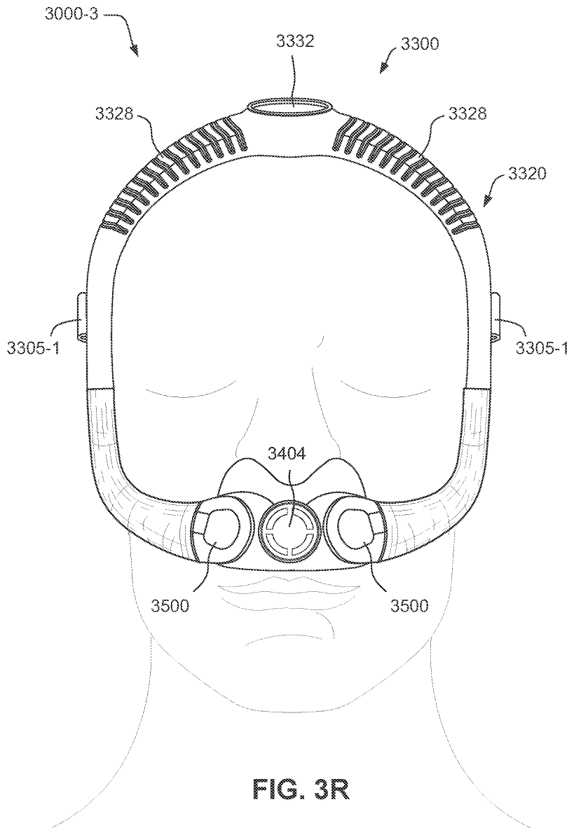

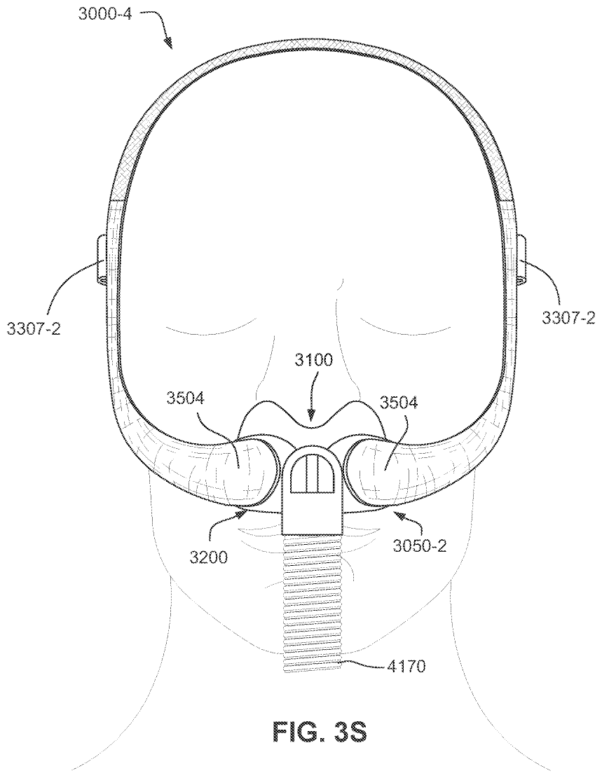

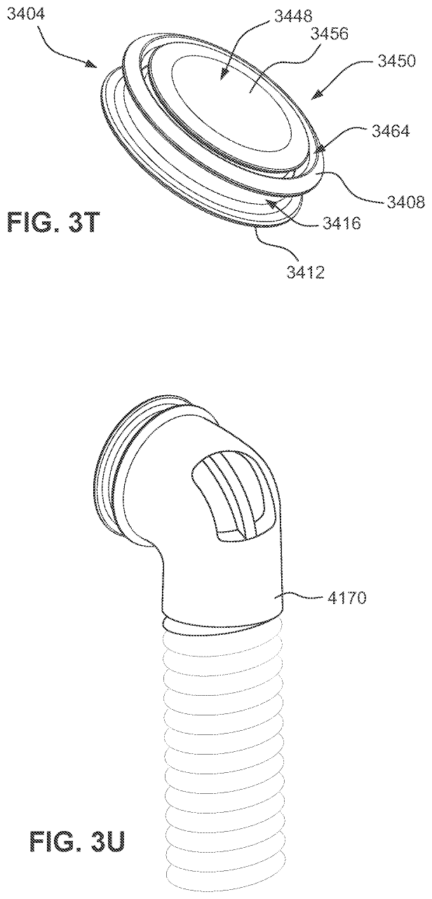

FIG. 3A shows a patient interface in the form of a nasal mask in accordance with one form of the present technology.

FIG. 3B shows a view of a plenum chamber 3200 showing a sagittal plane and a mid-contact plane.

FIG. 3C shows a view of a posterior of the plenum chamber of FIG. 3B. The direction of the view is normal to the mid-contact plane. The sagittal plane in FIG. 3C bisects the plenum chamber into left-hand and right-hand sides.

FIG. 3D shows a cross-section through the plenum chamber of FIG. 3C, the cross-section being taken at the sagittal plane shown in FIG. 3C. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3E shows the plenum chamber 3200 of FIG. 3B in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3E the plenum chamber 3200 is that of a nasal mask, and the superior point 3220 sits approximately on the sellion, while the inferior point 3230 sits on the lip superior.

FIG. 3F shows a patient interface having conduit headgear, in accordance with one form of the present technology.

FIG. 3G shows a perspective view of a cushion of a patient interface configured to be worn by a patient and convey pressurized air to the patient's nose and the patient's mouth.

FIG. 3H shows a perspective view of a cushion of a patient interface configured to be worn by a patient and convey pressurized air to the patient's nose.

FIG. 3I shows a perspective view of tubes usable with either the cushion of FIG. 3G or the cushion of FIG. 3H.

FIG. 3J shows a perspective view of rigidiser arms usable with either the cushion of FIG. 3G or the cushion of FIG. 3H.

FIG. 3K shows a perspective view of headgear straps usable with the cushion of FIG. 3G.

FIG. 3L shows a perspective view of headgear straps usable with the cushion of FIG. 3H.

FIG. 3M shows a front view of a pair of sleeves that is removably fitted to either the tubes of FIG. 3I or the rigidiser arms of FIG. 3J.

FIG. 3N shows a front view of a full sleeve that is removably fitted to the rigidiser arms of FIG. 3J.

FIG. 3O shows a front perspective view of yet another alternate form of a full sleeve that is removably fitted to the rigidiser arms of FIG. 3J.

FIG. 3P is a front view of a patient wearing the cushion of FIG. 3G connected to the tubes of FIG. 3I, the headgear straps of FIG. 3K, and the sleeves of FIG. 3M.

FIG. 3Q is a front view of a patient wearing the cushion of FIG. 3G connected to the rigidiser arms of FIG. 3J, the headgear straps of FIG. 3K, and the sleeve of FIG. 3N.

FIG. 3R is a front view of a patient wearing the cushion of FIG. 3H connected to the conduit headgear of FIG. 3I, and the headgear straps of FIG. 3L.

FIG. 3S is a front view of a patient wearing the cushion of FIG. 3H connected to the rigidisier arms of FIG. 3J, the headgear straps of FIG. 3L, and the sleeve of FIG. 3O.

FIG. 3T is a perspective view of an exemplary vent assembly.

FIG. 3U is a perspective view of an exemplary air circuit.

### 4.4 RPT Device

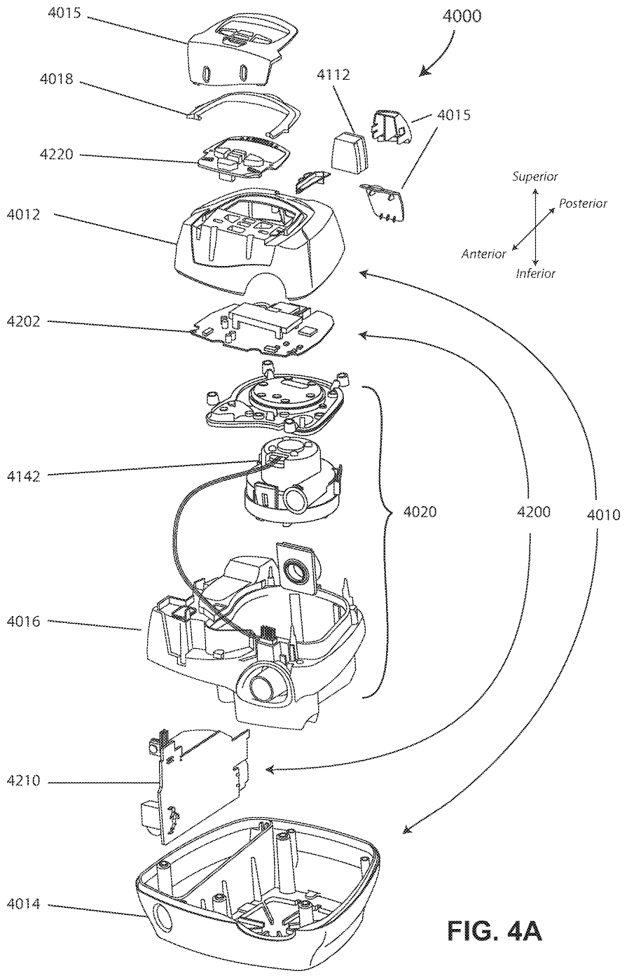

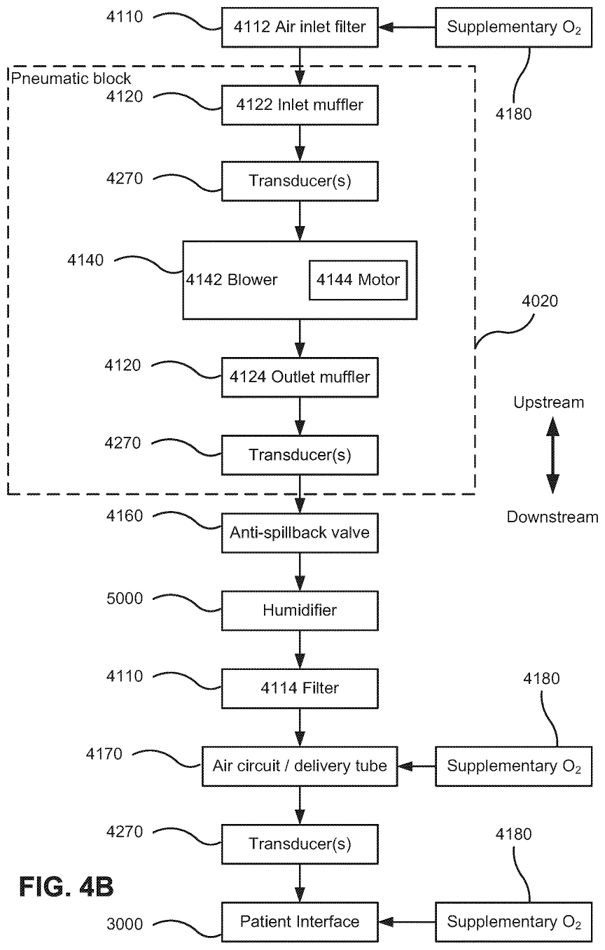

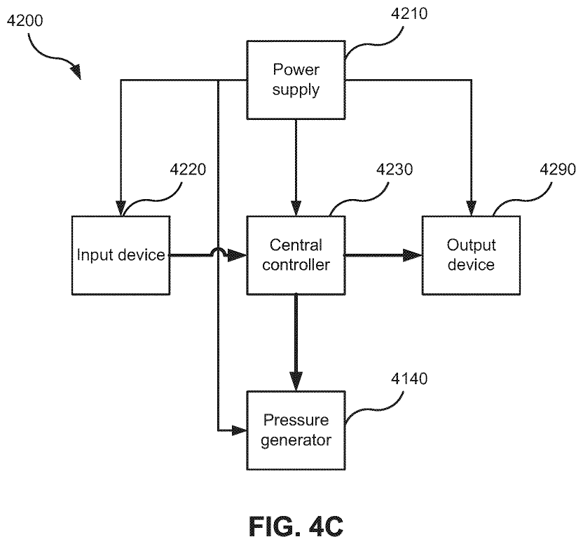

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram illustrating the interconnection of various electrical components of the RPT device.

### 4.5 Humidifier

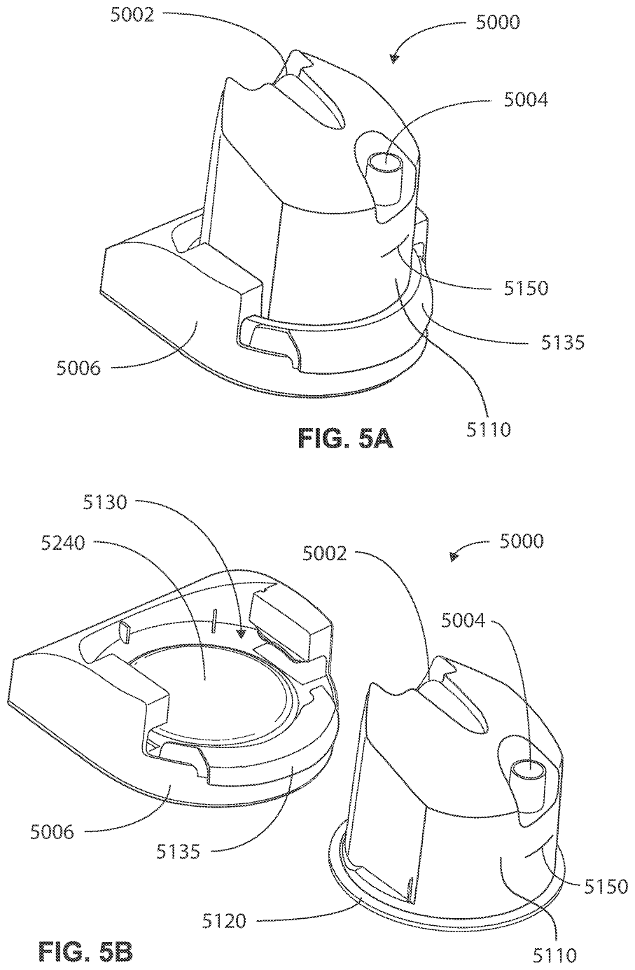

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Pneumatically Controlled Vent

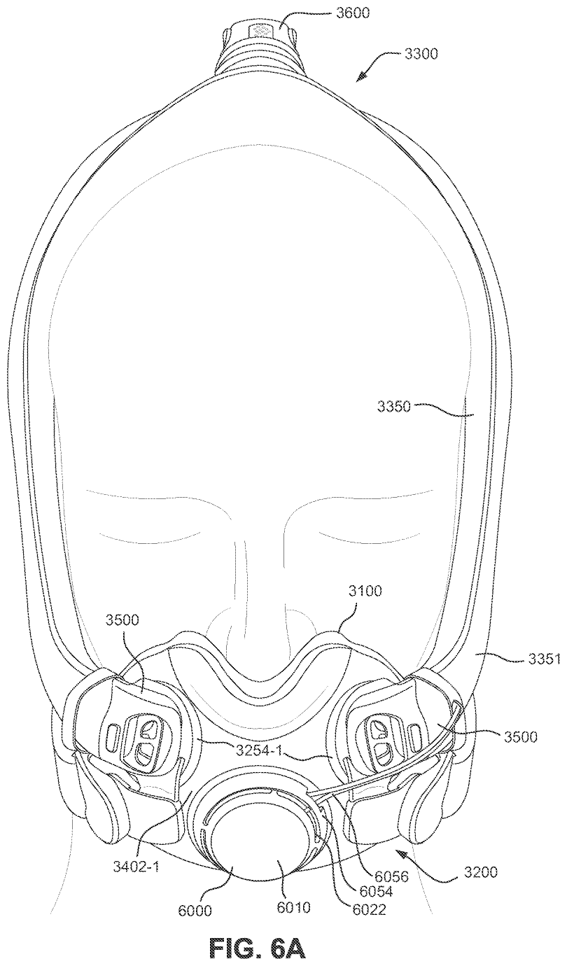

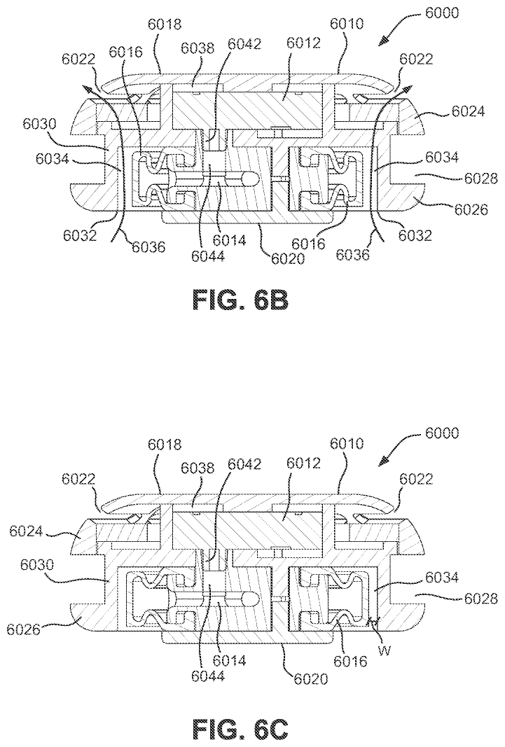

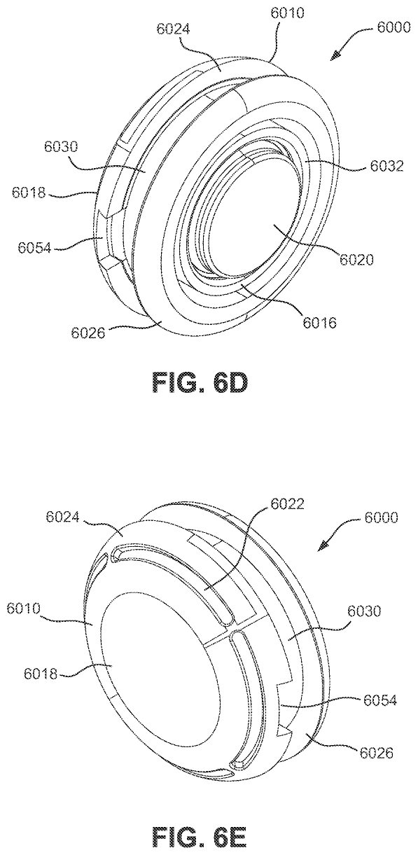

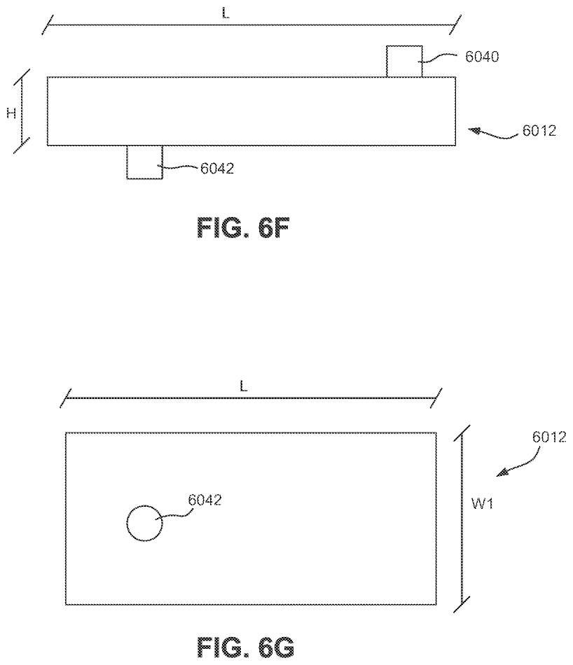

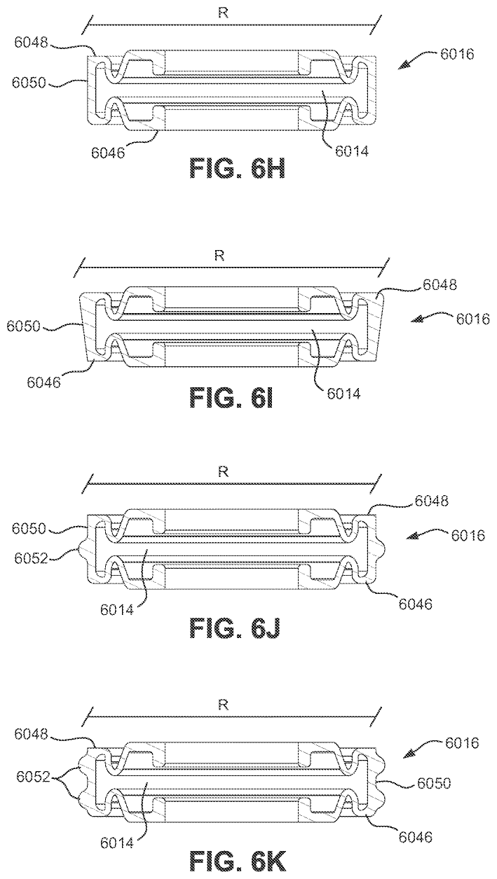

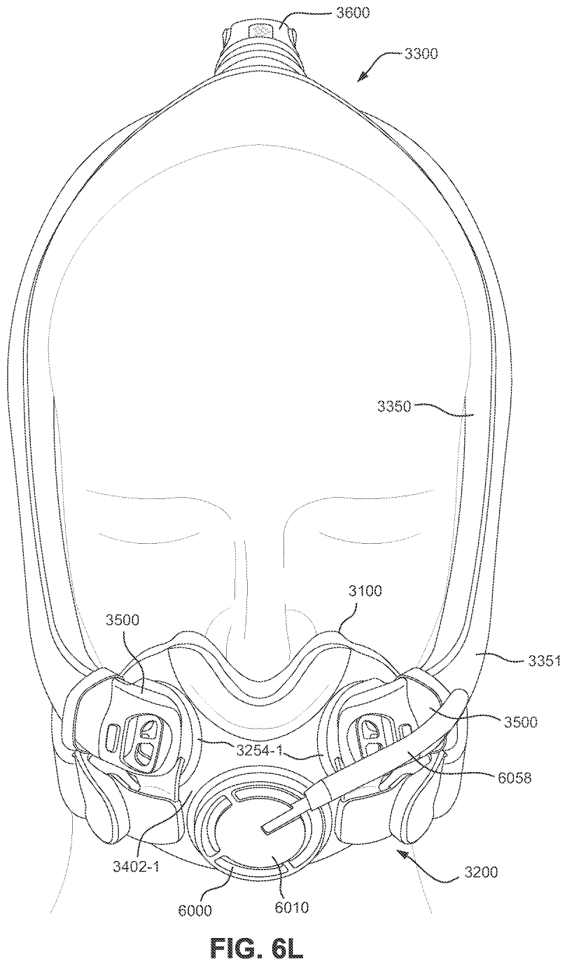

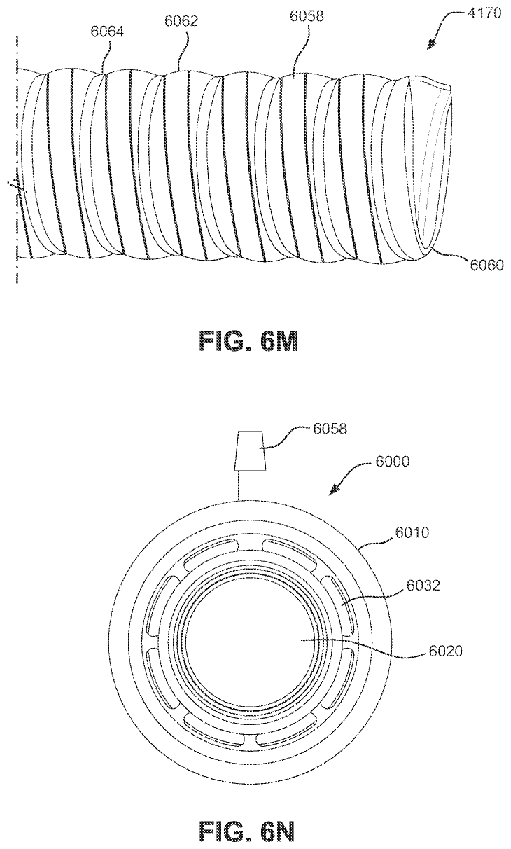

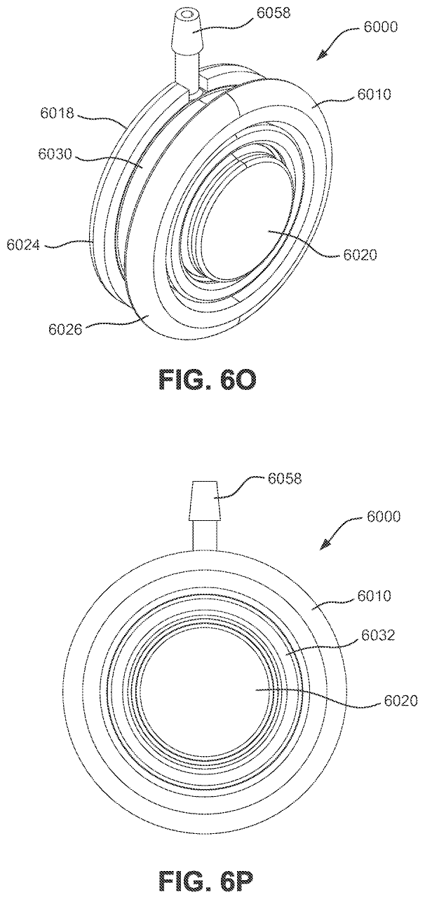

FIG. 6A shows a pneumatically controlled vent attached to a seal-forming structure.

FIG. 6B shows a cross-section view of the vent of FIG. 6A when the inflatable body is deflated.

FIG. 6C shows a cross-section view of the vent of FIG. 6A when the inflatable body is inflated.

FIG. 6D shows a perspective view of the vent of FIG. 6A.

FIG. 6E shows another perspective view of the vent of 6A.

FIGS. 6F and 6G show a schematic of an exemplary pump.

FIGS. 6H-K show inflatable bodies with various geometries.

FIG. 6L shows another pneumatically controlled vent attached to a seal-forming structure.

FIG. 6M shows a pneumatic conduit wrapped around an air delivery conduit.

FIG. 6N shows a perspective view of the vent of FIG. 6L.

FIG. 6O shows another perspective view of the vent of FIG. 6L.

FIG. 6P shows another perspective view of the vent of FIG. 6L.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3F, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components (e.g., a cushion module 3150 together with the seal-forming structure 3100) in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH₂O with respect to ambient.

### 5.3.1 Seal-Forming Structure

In one form of the present technology, a seal-forming structure 3100 provides a target seal-forming region and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example, the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200 and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.1.6 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3F, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.1.7 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

### 5.3.2.1 Multiple Openings

As shown in FIGS. 3G and 3H, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear-described below) or a “tube-down” configuration (e.g., using a single conduit in front of the patient's face).

### 5.3.2.1.1 Nose and Mouth Mask

As shown in FIG. 3G, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 3G). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 5.3.2.1.2 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 3H, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 3H). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. An example of a positioning and stabilising structure is shown in FIG. 3A.

In one form the positioning and stabilising structure 3300 provides a retention force at least sufficient to overcome the effect of the positive pressure in the plenum chamber 3200 to lift off the face (i.e., Fₚₗₑₙᵤₘ).

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. suitable for a small sized head, but not a large sized head.

### 5.3.3.1 Conduit Headgear

### 5.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3F, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3F, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 7C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 3Z, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

### 5.3.3.1.2 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3F comprises tubes 3350, the superior portions of which comprise extendable tube sections each in the form of an extendable concertina structure 3362.

In some forms, the extendable concertina structure 3328 may be formed as a series of ridges and grooves on the surface of the tubes 3350. The concertina structure 3328 may be biased toward a retracted position and may move to an expanded position when the patient dons the positioning and stabilising structure 3300. Because portions of the tubes 3350 may be substantially inextensible (e.g., non-extendable tube sections 3363), the concertina structures 3328 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3328. Alternatively, the tubes 3350 may be manufactured in multiple sizes (e.g., small, medium, large). The patient may select a length that most closely conforms to their head, and the concertina structures 3328 may make small adjustments in order to tailor the fit to the individual patient.

In some forms, the inlet 3332 may be disposed in the middle of the conduit 6320. For example, the tubes 3350 may be symmetric about the inlet 3332 through at least one axis.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connect to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal forming structure 3100. The tubes 3350 may contact the patient's face below the patient's cheekbones, which may be more comfortable than contact on the patient's cheekbones and may avoid excessively obscuring the patient's peripheral vision.

### 5.3.3.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3F, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.3.1.4 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.3.2 Headgear Straps

In some forms, the positioning and stabilising structure 3300 may include headgear 3302 with at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

Two forms of the headgear, four-point headgear 3302-1 and two-point headgear 3302-2, are discussed in more detail below as illustrative examples.

### 5.3.3.2.1 Four-Point Connection

As shown in FIG. 3K, some forms of the headgear 3302-1 may be a four-point connection headgear. This means that the headgear 3302-1 may connect to four separate places on the plenum chamber 3200, on a frame connected to the plenum chamber 3200, and/or on arms connected to the plenum chamber 3200. The headgear 3302-1 may include four different straps providing a tensile force to help maintain the seal-forming structure 3100 in a sealing position. The positioning and stabilising structure 3300 of FIG. 3A may also be considered a four-point connection headgear.

In some forms, the headgear 3302-1 may include inferior straps 3304-1, which may connect to an inferior portion of the cushion 3050-1. The inferior straps 3304-1 may extend along the patient's cheek toward a posterior region of the patient's head. For example, the inferior straps 3304-1 may overlay the masseter muscle on either side of the patient's face. The inferior straps 3304-1 may therefore contact the patient's head below the patient's ears. The inferior straps 3304-1 may meet at the posterior of the patient's head, and may overlay the occipital bone and/or the trapezius muscle.

The headgear 3302-1 may also include superior straps 3305-1, which may overlay the temporal bones, parietal bone, and/or occipital bone. The superior straps 3305-1 may also connect to the tubes 3350 (e.g., by interfacing with the tabs 3320).

A rear strap 3307-1 may extend between the superior straps 3305-1 and between the inferior straps 3304-1. The inferior and superior straps 3304-1, 3305-1 on a given side (e.g., left or right) may also be connected to the rear strap 3307-1 adjacent to one another. The height of the rear strap 3307-1 may therefore be approximately the combined height of the inferior and superior strap 3304-1, 3305-1. The rear strap 3307-1 may overlay the occipital bone and/or the pariental bone in use. This may allow the rear strap 3307-1 to assist in anchoring the headgear 3302-1 to the patient's head.

In the illustrated example, the headgear 3302-1 may be formed with a substantially X-shape. The inferior and superior straps 3304-1, 3305-1 may be connected to a rear strap 3307-1 using stitching, ultrasonic welding, or any similar process.

In some forms, the inferior straps 3304-1 are connected to a magnetic member 3306-1. For example, each inferior straps 3304-1 may be threaded through a magnetic member 3306-1, so that a length of each inferior strap 3304-1 may be adjusted. The magnetic members 3306-1 may removably connect to the magnets 3370-1 (described below), so that the inferior straps 3304-1 may be disconnected from the plenum chamber 3200, but the length of the inferior straps 3304-1 may not be affected.

In some forms, the superior straps 3305-1 may be connected directly to the tabs 3320 of the tubes 3350. The superior straps 3305-1 may be threaded through the tabs 3320 in order to adjust the length and control the tensile force of each superior strap 3305-1.

In some forms, the headgear 3302-1 may be used only with the nose and mouth cushion 3050-1 (e.g., because the nose-only cushion 3050-1 does not have four connection points). However, the headgear 3302-1 may be used interchangeably with the tubes 3350 and the rigidiser arms 3340.

### 5.3.3.2.2 Two-Point Connection

As shown in FIG. 3L, some forms of the headgear 3302-2 may be a two-point connection headgear. This means that the headgear 3302-2 may connect to two separate places.

In some forms, the headgear 3302-2 may be formed from a continuous piece of material. In other words, the headgear 3302-2 may not be formed from multiple straps connected (e.g., stitched) together. This may be comfortable for a patient as they will not be in contact with any seams or joints connecting different straps. In other forms, the headgear 3302-2 may be formed from multiple straps (e.g., two superior straps, a rear strap, etc.) that are connected together (e.g., with stitching, ultra-sonic welding, etc.).

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3F, the patient interface 3000 comprises a strap 3307-2 forming part of the positioning and stabilising structure 3300. The strap 3307-2 may be known as a back strap or a rear headgear strap, for example. The rear strap 3307-2 may overlay the temporal bones, parietal bone, and/or occipital bone. In other examples of the present technology, one or more further straps may be provided. For example, patient interfaces 3000 according to examples of the present technology having a nose-and-mouth cushion may have a second, lower, strap configured to lie against the patient's head proximate the patient's neck and/or against posterior surfaces of the patient's neck.

In the example shown in FIG. 3F, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3Z, the positioning and stabilising structure 3300 comprises a pair of tabs 3320. In use a strap 3310 may be connected between the tabs 3320. The strap 3310 may be sufficiently flexible to pass around the back of the patient's head and lie comfortably against the patient's head, even when under tension in use.

As shown in FIG. 3L, some forms of the headgear 3302-2 may be at least partially bifurcated. For example, a rear strap 3307-2 of the headgear 3302-2 (e.g., configured to contact the posterior portion of the patient's head) may be wider than the surrounding portions of the headgear 3302-2. An intermediate section 3308-2 of the rear strap 3307-2 may include a slit 3309-2. A superior section of the rear strap 3307-2 may therefore be movable relative to the inferior section as a result of the slit 3309-2. This may allow the patient to have a larger strap coverage on the posterior region of their head, which may assist in better anchoring the headgear 3302-2 to the patient's head since there is no inferior strap (e.g., 3304-1).

In some forms, the headgear 3302-2 may be used only with the nasal cushion 3050-2 (e.g., because the nose and mouth cushion 3050-1 does not have four connection points). However, the headgear 3302-2 may be used interchangeably with the tubes 3350 and the rigidiser arms 3340.

### 5.3.3.3 Rigidiser Arm

As shown in FIG. 3J, a rigidiser arm 3340 may be an elongated, rigid member that assists in maintaining the cushion (e.g., the nose and mouth cushion 3050-1 or the nasal cushion 3050-2) in an operating position. The rigidiser arm 3340 may contact a side of the patient's head and provide a force to limit slipping of the seal-forming structure 3100 from the patient's nose and/or mouth.

In some forms, the rigidiser arm 3340 is constructed from a rigid material (e.g., plastic). The rigid material may not permit the rigidiser arm 3340 to stretch. Additionally, the rigidiser arm 3340 may be substantially inflexible and may be unable to bend. The rigidiser arm 3340 may be pre-molded into a desired shape in order to fit a patient's head. For example, the rigidiser arms 3340 may be molded with a curved shape to substantially correspond to the shape of the side of the patient's head (e.g., overlaying the masseter muscle and/or the temporal bone).

In certain forms, the rigidiser arm 3340 may be molded in order to conform to a specific patient's head (e.g., the rigidiser arm 3340 is customized).

In some forms, the rigidiser arm 3340 may be flexible along at least one direction. For example, the rigidiser arm 3340 may be flexible about its width and may be inflexible along its length. In other words, the rigidiser arm 3340 may be bendable about an axis along the width of the rigidiser arm 3340, but may be unable to bend about an axis perpendicular to the rigidiser arm 3340. This may allow an individual patient to adjust the rigidiser arm 3340 in order to better fit their individual head.

In certain forms, the rigidiser arm 3340 may remain in the new position after being bent. This may allow a patient adjust the shape of the rigidiser arm 3340 for their specific head and then the rigidiser arm 3340 will keep the desired shape while in use in order to promote patient comfort.

In some forms, a first end 3342 of the rigidiser arm 3340 may be a free end and a second end 3344 (e.g., opposite of the first end 3342) of the rigidiser arm 3340 may be fixed. The first end 3342 may be curved in order to minimize sharp edges that could cause patient discomfort. The first end 3342 may also overlay the patient's head proximate to the temporal bone, in use. The second end 3344 may be fixed to an arm connection structure 3504.

In some forms, the arm connection structure 3504 may be similar to the conduit connection structure 3500. For example, the arm connection structure 3504 and the conduit connection structure 3500 may have substantially the same shape. This may allow either the conduit connection structure 3500 or the arm connection structure 3504 to fit into the groove (e.g., 3266-1 or 3266-2) and connect to the plenum chamber inlet port 3254. The arm connection structure 3504 may connect to the nose and mouth cushion 3050-1 or the nose-only cushion 3050-2 in substantially the same way as the conduit connection structure 3500 (e.g., via a snap fit, press fit, friction fit, etc.).

In some forms, the arm connection structure 3504 may act as a plug for the plenum chamber inlet port 3254 (e.g., either 3254-1 and/or 3254-2). Unlike the tubes 3350, the rigidiser arm 3340 does not convey pressurized air to the plenum chamber 3200. The rigidised arm 3340 may be used with a “tube down” configuration, where a hose is connected to the vent opening 3402 (e.g., either 3402-1 and/or 3402-2), and conveys air into the plenum chamber 3200 through the vent opening 3402. In this example, air does not need to travel into or out of the plenum chamber inlet openings 3254. Thus, the arm connection structure 3504 may form a seal with the plenum chamber inlet opening 3254 in order to limit airflow into or out of the plenum chamber 3200.

### 5.3.3.4 Sleeve

In some forms, to allow for modularity, a sleeve may be used with the tubes 3350 and/or the rigidisier arms 3340. The sleeve may at least partially surround the tubes 3350 and/or the rigidiser arms 3340. As shown in FIGS. 3M to 3O, different shapes of sleeves may be used, which may correspond to different types of positioning and stabilising structures 3300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use and may feel soft against the patient's skin in order to improve patient compliance.

The material may also be flexible in order to assist in donning or doffing the sleeve from the tube 3350 or the rigidiser arms 3340. For example, the material may allow the sleeve to bend in order to conform to the shape of the tubes or conduit headgear 3350 or the rigidiser arms 3340, which may change depending on the shape of an individual patient's head.

In some forms, the sleeve may also be at least partially elastic (e.g., the material may allow the sleeve to stretch). The elastic material may help the sleeve stretch in order to fit around the tubes 3350 or the rigidiser arms 3340. The elastic material may then return to an initial position that is snug against the tubes 3350 or the rigidiser arms 3340 in order to limit the sleeve from sliding while in use.

### 5.3.3.4.1 Conduit Sleeve

As shown in FIG. 3M, one example of a sleeve is a conduit sleeve 3351, which may be usable with the tubes 3350 described above.

As shown in FIG. 3M, the conduit sleeve 3351 may include a curved shape that may be similar to the shape of the tubes 3350 shown in FIG. 3I. The flexible material used to construct the conduit sleeve 3351 may allow the conduit sleeve 3351 to further curve in order to correspond to the shape of the tubes 3350 (e.g., when worn by the patient).

In some forms, the conduit sleeve 3351 may include a first or superior opening 3352. The superior opening 3352 may be disposed at one end of the conduit sleeve 3351. The superior opening 3352 may be an opening to a passage that extends along at least a portion of the conduit sleeve 3351.

As shown in FIG. 3M, some forms of the conduit sleeve 3351 may also include an inferior extension 3354. The inferior extension 3354 may be positioned on an opposite end of the conduit sleeve 3351 from the superior opening 3352. The conduit sleeve 3351 may be customized to fit a particular user's face. For instance, the inferior extension 3354 of the conduit sleeve 3351 may be configured in a relatively more posterior region or anterior region of the patient's head.

Some forms of the inferior extension 3354 may include a rigid or semi-rigid piece (e.g., within the sleeve 3351). The rigid or semi-rigid piece may be constructed from a plastic material, or a similar material. Alternatively, the inferior extension 3354 may be stiffened using a manufacturing process (e.g., stitching rigidised thread, flat knitting, using thicker material).

As shown in FIG. 3M, some forms of the inferior extension 3354 may include a connection member 3356. In the illustrated example, the connection member 3356 may be a magnet, although in other examples, the connection member 3356 may be a different type of connector (e.g., a mechanical fastener, an adhesive, hook and loop material, etc.). The connection member 3356 may also be positioned at an end of the inferior extension 3354, although the connection member 3356 could alternatively be positioned anywhere along the inferior extension 3354.

In some forms, the connection member 3356 (e.g., a magnet) may be removably connected to the magnets 3370-1 of the headgear 3302-1. For example, when the conduit sleeves 3351 are connected to the tubes 3350 (see e.g., FIG. 3P), the magnets 3370-1 connected to the inferior straps 3304-1 may be removably connected to the connection member 3356 in order to provide the tensile force.

### 5.3.3.4.2 Four-Point Arm Sleeve

As shown in FIG. 3N, another example of a sleeve is a four-point arm sleeve 3380, which may be usable with the rigidiser arms 3340 described above.

As shown in FIG. 3N, the four-point arm sleeve 3380 may include a curved shape that may be similar to the shape of the rigidiser arm 3340 shown in FIG. 3J. The flexible material used to construct the four-point arm sleeve 3380 may allow the four-point arm sleeve 3380 to further curve in order to correspond to the shape of the rigidiser arm 3340 (e.g., when worn by the patient and/or went bent by the patient).

As shown in FIG. 3N, some forms of the four-point arm sleeve 3380 may include an inferior extension 3384. The inferior extension 3384 may be positioned at an end of the four-point arm sleeve 3380.

In the illustrated example, the shape and/or structure of the inferior extension 3384 is substantially the same as the shape of the inferior extension 3354. For example, the inferior extension 3384 may be more rigid as compared to the rest of the four-point arm sleeve 3380 (e.g., as a result of rigidising thread or rigid material).

As shown in FIG. 3N, some forms of the inferior extension 3384 may include a connection member 3386. In the illustrated example, the connection member 3386 may be a magnet, although in other examples, the connection member 3386 may be a different type of connector (e.g., a mechanical fastener, an adhesive, hook and loop material, etc.). The connection member 3386 may also be positioned at an end of the inferior extension 3384, although the connection member 3386 could alternatively be positioned anywhere along the inferior extension 3384.

In some forms, the connection member 3386 (e.g., a magnet) may be removably connected to the magnets 3370-1 of the headgear 3302-1. For example, when the four-point arm sleeves 3380 are connected to the rigidiser arm 3340 (see e.g., FIG. 3Q), the magnets 3370-1 connected to the inferior straps 3304-1 may be removably connected to the connection member 3386 in order to provide the tensile force.

As shown in FIG. 3N, the four-point arm sleeve 3380 may include a pair of tabs 3394, which may be similar to the tab 3320 on the tubes 3350. When the four-point arm sleeve 3380 is worn by the patient, the tabs 3394 may be positioned in substantially the same place on the patient's head as where the tabs 3320 are positioned when the patient wears the tubes 3350.

### 5.3.3.4.3 Two-Point Arm Sleeve

As shown in FIG. 3O, yet another example of a sleeve is a two-point arm sleeve 3380-1, which may be usable with the rigidiser arms 3340 described above.

In some forms, the two-point arm sleeve 3380-1 may be similar to the four-point arm sleeve 3380 described above. Only some similarities and differences may be described below.

As shown in FIG. 3O, the two-point arm sleeve 3380-1 may include an inferior opening 3388-1 that is positioned at an end of the two-point arm sleeve 3380-1. The inferior opening 3388-1 may form an opening to a passageway through the two-point arm sleeve 3380-1. In the illustrated example, the inferior opening 3388-1 may open into a surface of the conduit sleeve 3380-1.

As shown in FIG. 3O, the two-point arm sleeve 3380-1 may include a pair of tabs 3394-1, which may be similar to the tab 3320 on the tubes 3350. When the two-point arm sleeve 3380-1 is worn by the patient, the tabs 3394-1 may be positioned in substantially the same place on the patient's head as where the tabs 3320 are positioned when the patient wears the tubes 3350.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO₂ by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

As shown in FIG. 3T, a vent 3450 may be used with the patient interface 3000. The vent 3450 may have a substantially similar shape to the vent opening 3402-1 (e.g., a substantially circular shape).

The vent 3450 may be used with either the mouth and nose plenum chamber 3200-1 (e.g., illustrated in FIG. 3G) or the nose-only plenum chamber 3200-2 (e.g., illustrated in FIG. 3H).

With reference to FIG. 3T, the vent 3450 may include a vent housing 3404, which may be configured to engage with the vent opening 3402. The vent housing 3404 may be constructed from a rigid material or a semi-rigid material. For example, the vent housing 3404 may be constructed from plastic, metal, or any similar material. The vent housing 3404 may add rigidity to the patient interface 3000 (e.g., to limit unwanted bending that may affect the position of the seal-forming structure 3100 on the patient's face).

The vent housing 3404 may include an anterior surface 3408, a posterior surface 3412, and a groove 3416. The anterior surface 3408 faces away from the patient's face in use and may be positioned outside the pressurized volume of the plenum chamber 3200. The posterior surface 3412 is disposed opposite to the anterior surface 3408. In use, the posterior surface 3412 may face the patient and may be disposed within the pressurized volume of the plenum chamber 3200. The groove 3416 may be formed between the anterior and posterior surfaces 3408, 3412. A portion of the plenum chamber 3200 may be received within the groove 3416 in order to retain the vent 3400 in position.

In some forms, a diffuser 3448 may be used with the vent housing 3404. The diffuser 3448 may assist with limiting the decibel output from any of the patient interface 3000 (or any other patient interface). Specifically, the diffuser 3448 may assist in limiting the decibel level associated with air output from the patient interface 3000 (e.g., exhaled air), although the diffuser 3448 may limit the decibel level of at any point in the patient interface.

In certain forms, the diffuser 3448 may diffuse, and therefore slow, the exhaust gas exiting the plenum chamber 3200 and passing through the vent housing 3404. The diffuser 3448 may assist in avoiding jetting and associated discomfort to the patient and/or bed partner (e.g., noise caused by jetting against a pillow, sheets, bedclothes, etc.).

In some forms, the diffuser may include an anterior surface 3456 that faces away from the patient in use. An outer diameter of the anterior surface 3456 may be less than an inner diameter of the vent housing 3404 proximate to the anterior surface 3408. This may form a gap 3464 through which air may travel.

### 5.3.4.1 Pneumatically Controlled Vent

It is contemplated that the vent 3400 may be in the form of a pneumatically controlled vent and/or may comprise a pneumatically controlled valve. The pneumatically controlled vent may be configured to control a flow rate of the gas being vented from a respiratory treatment system.

It may be advantageous for a respiratory treatment system to be able to pneumatically control a flow rate of gas being vented therefrom. For example, a pneumatically controlled vent would be able to actively control CO₂ washout throughout a therapy session. This would allow the system to tailor the CO₂ washout to the unique washout needs of each individual user, thereby improving comfort and sleep continuity.

FIG. 6A shows an exemplary pneumatically controlled vent 6000 positioned within the vent opening 3402-1 in the oronasal seal-forming structure (patient interface or patient interface assembly) 3100. As can be seen, the pneumatically controlled vent 6000 may be positioned between the plenum chamber inlet ports 3254-1. It is contemplated that the pneumatically controlled vent 6000 can be located in other positions on the seal-forming structure 3100. For example, in configurations utilizing only one inlet port, the pneumatically controlled vent 6000 may be located above, below or lateral to the inlet port. In addition, the pneumatically controlled vent 6000 may be permanently or removably secured within the vent opening 3402-1. It should be understood that the pneumatically controlled vent 6000 may be used with any type of seal-forming structure with an interface that is able to receive the vent 6000 including the nasal seal-forming structure 3100 that comprises the vent opening 3402-2.

As illustrated in FIGS. 6B-E, the pneumatically controlled vent 6000 may include a housing 6010, a pump 6012, an air chamber 6014, and an inflatable body 6016. In addition, the air chamber 6014, and the inflatable body 6016 may be pneumatically sealed off from atmosphere so that pressure in the inflatable body 6016 can be selectively maintained above ambient pressure. However, when the pump 6012 is deactivated or inoperable, the gas in the inflatable body 6016 and the air chamber 6014 may be allowed to be vented to atmosphere in order to equalize the pressure in the inflatable body 6016 with respect to ambient pressure.

The housing 6010 may enclose and form the structural support for the internal components of the pneumatically controlled vent 6000. The housing 6010 may include a cap (or outer) portion 6018 and a base (or inner) portion 6020. It is contemplated that the cap portion 6018 and the base portion 6020 may be a unitarily formed body. Alternatively, the cap portion 6018 and the base portion 6020 may be separately formed and assembled together by way of bonding, adhesion, mechanical fasteners, etc. In addition, all of the portions of the housing 6010 may be formed from a relatively rigid material such as, for example, polypropylene, polystyrene, polycarbonate, etc. Alternatively, the housing 6010 may be formed from relatively flexible material such as, for example, silicone. In addition, the different portions of the housing 6010 may be formed from different materials so that different portions of the housing 6010 have different rigidities.

The cap portion 6018 may include one or more outlet openings 6022 through which the gas washout is vented to atmosphere. The outlet openings 6022 may be arranged around a periphery of the cap portion 6018 so that the outlet openings 6022 form a circumferential ring around a central portion of the cap portion 6018. When there is a plurality of outlet openings 6022, the outlet openings 6022 may all have the same size and shape. Alternatively, each outlet opening 6022 may have a different size and/or shape from other outlet openings 6022. In addition, although the outlet openings 6022 are shown in a symmetrical arrangement (e.g., number of openings or opening size evenly distributed on the cap portion 6018), the arrangement of outlet openings 6022 may be asymmetrical (e.g., number of openings or opening size unevenly distributed on the cap portion 6018).

The cap portion 6018 may also include a pair of flanges 6024 and 6026 that together form a channel 6028 to secure the pneumatically controlled vent 6000 to the seal-forming structure 3100. The flanges 6024 and 6026 may extend radially outward from a circumferential wall 6030 of the cap portion 6018 so that the channel 6028 is open in a radially outward direction. In addition, the channel 6028 may receive a rim of the vent opening 3402-1 (or 3402-2) of the seal-forming structure 3100. In order to secure the rim of the vent opening 3402-1 (or 3402-2) within the channel 6028, the diameter of the flanges 6024 and 6026 may be larger than the diameter of the vent opening 3402-1 (or 3402-2).

It is contemplated that for configurations in which the pneumatically controlled vent 6000 is permanently attached to the seal-forming structure 3100, the housing 6010 (or at least the cap portion 6018 may be an integral part of a plenum wall of the seal-forming structure 3100. In such a configuration, the flanges 6024 and 6026 and the channel 6028 may be replaced with the rim of the vent opening 3402-1 or 3402-2.

The base portion 6020 of the housing 6010 may form the innermost portion of the housing 6010 and may be positioned within the plenum chamber 3200. The base portion 6020 and the cap portion 6018 together may form one or more inlet openings 6032 of one or more air passages 6034 that convey gas washout through the pneumatically controlled vent 6000. In addition, the inflatable body may be secured to the base portion 6020.

The one or more air passages 6034 may extend from the one or more inlet openings 6032 to the one or more outlet openings 6022. In addition, the one or more inlet openings 6032 may be arranged around a periphery of the base portion 6020 so that the inlet openings 6032 form a circumferential ring around the base portion 6020. When there is a plurality of inlet openings 6032, the inlet openings 6032 may all have the same size and shape. Alternatively, each inlet opening 6032 may have a different size and/or shape from other inlet openings 6032.

It is contemplated that the one or more inlet openings 6032 may be distributed in a symmetrical arrangement (e.g., number of openings or opening size evenly distributed around the base portion 6020) or in an asymmetrical arrangement (e.g., number of openings or opening size unevenly distributed around the base portion 6020). The arrangement of the one or more inlet openings 6032 may match or not match the arrangement of the one or more outlet openings 6022. FIGS. 6A, 6C, and 6D show an exemplary configuration in which the one or more inlet openings 6032 do not match the arrangement of the one or more outlet openings 6022. The illustrated arrangements show a single inlet opening 6032 around the perimeter of the base portion 6020 and a plurality of evenly spaced outlet openings 6022 that are equally sized.

As illustrated in FIG. 6B, a flow 6036 of gas washout may enter the air passage 6034 through the one or more inlet openings 6032 and exit the pneumatically controlled vent 6000 through the one or more outlet openings 6022. In the illustrated configuration, the air passage 6034 is a ring-shaped gap extending from an inlet side of the vent 6000 to an outlet side of the vent 6000. However, the air passage 6034 may have any form capable of conveying the gas washout from the plenum chamber 3200 to atmosphere. As shown in FIG. 6C, the air passage 6034 has a cross-sectional width W. The flow rate of the gas washout flowing through the air passage 6034 may be regulated by adjusting the cross-sectional width W of at least a portion of the air passage 6034 (e.g., widening and narrowing the ring-shaped gap). This may be achieved by actuating the pump 6012 to inflate and deflate the inflatable body 6016. It is contemplated that the inflatable body 6016 may be inflated and/or deflated incrementally or dynamically. The pressure of the gas inflating the inflatable body 6016 may be greater than, less than, or the same as the pressure of the gas washout flowing through the vent 6000 and/or the pressure of gas in the seal-forming structure 3100.

In addition, the inflation and deflation of the inflatable body 6016 may be coordinated or synchronized with the ramping up and ramping down of the treatment pressure. In addition, the inflatable body 6016 and/or the pump 6012 may be configured to allow a maximum of 20% rebreathing of exhaled gas regardless of the venting demand or requirement. The inflatable body 6016 and/or the pump 6012 may also be configured to account for leakage in the seal-forming portion 3100.

The pump 6012 may be located within a chamber 6038 inside the cap portion 6018 and may be any type of compact pump that can fit within the dimensions of the chamber 6038. For example, the pump 6012 may be a diaphragm pump, a piezoelectric pump, or a peristaltic pump. It is contemplated that a width W1 of the pump 6012 may be about 14 mm to about 16 mm. A length L of the pump 6012 may be about 14 mm to about 16 mm. In addition, a height H of the pump 6012 may be about 3 mm to about 5 mm.

When actuated, the pump 6012 may draw air from atmosphere through an inlet 6040 and may discharge pressurized air to the air chamber 6014 through an outlet 6042. When disabled or not actuated, air may be allowed to flow through the pump 6012 from the outlet 6042 to the inlet 6040 and out to atmosphere. It is contemplated that the inlet 6040 may extend through the cap portion 6018 of the housing 6010 or may be fluidly connected to the outlet openings 6022. It is further contemplated that the inlet 6040 may be fluidly connected to a relief valve or may itself be in the form of a relief valve so that the pressure in the inflatable body 6016 may be equalized with ambient pressure when gas flow to the vent 6000 is terminated.

In addition, although FIG. 6F shows the inlet 6040 projecting from the main body of the pump 6012, it should be understood that the inlet 6040 may be in any form that allows gas to be draw in to the pump 6012. For example, the inlet 6040 may be in the form of a nozzle or in the form of an opening or inlet hole in the main body of the pump 6012. When the inlet 6040 is in the form of an inlet hole, the inlet hole may be open to atmosphere through gaps in the chamber 6038 that are in communication with the outside of the vent 6000 either through the outlet openings 6022 or other openings in the cap portion 6018. In other words, the inlet 6040 does not necessarily have to extend through the cap portion 6018 to draw atmospheric air into the pump 6012.

The outlet 6042 of the pump 6012 is pneumatically connected to the air chamber 6014 by way of an air passage 6044. The air passage 6044 may be located at a central part of the air chamber 6014 or at any location radially inward from the perimeter of the air chamber 6014. The perimeter of the air chamber 6014 may be bound by the inflatable body 6016 so that air flowing radially outward from the air passage 6044 enters the inflatable body 6016. Alternatively, the outlet 6042 of the pump 6012 may be directly attached to the air chamber 6014.

The inflatable body 6016 may be in the form of a ring that surrounds the air chamber 6014. In addition, the inflatable body 6016 may be formed from an elastomeric material such as, for example, silicone rubber. The elastomeric material allows the inflatable body 6016 to inflate and expand in size when filled with the pressurized air from the pump 6012. The inflatable body 6016 may be configured to expand radially outward when being filled with the pressurized air from the pump 6012. For example, the inflatable body 6016 may include a gusset or concertina configuration.

At least a portion of the air passage 6034 may be formed by a gap between the perimeter of the inflatable body 6016 and the circumferential wall 6030 (or other exterior wall) of the cap portion 6018 of the housing 6010. In this configuration, the cross-sectional width W of the air passage 6034 may be at its maximum when the pump 6012 is not active or disabled and the pressure in the air chamber 6014 and the inflatable body 6016 is substantially the same pressure as atmosphere. Conversely, the cross-sectional width W of the air passage 6034 may be smallest when the pump 6012 is actuated to generate a maximum pressure that causes the inflatable body 6016 to radially expand to its maximum extent.

FIG. 6B shows a configuration in which the inflatable body 6016 is deflated to its minimum radial diameter (i.e., not radially expanded) and the pressure in the inflatable body 6016 is substantially the same as atmosphere. FIG. 6C shows a configuration in which the inflatable body 6016 is radially expanded to its maximum extent (the pressure inside the inflatable body 6016 is at its maximum). It is contemplated that there still may be a gap between the circumferential wall 6030 (or other external wall) when the inflatable body 6016 is inflated to its maximum extent so that gas washout may still be vented to atmosphere. Alternatively, the inflatable body 6016 may completely close the air passage 6034 when the inflatable body 6016 is inflated to its maximum extent so that the vent 6000 does not vent any gas washout.

FIGS. 6H-6K illustrate different variations of the inflatable body 6016. It should be understood that the variations illustrated in FIGS. 6H-6K are exemplary and that the shape of the inflatable body 6016 is not limited to those variations illustrated in FIGS. 6H-6K.

FIG. 6H illustrates an inflatable body 6016 in which the radius R is the same throughout the cross-section of the inflatable body 6016. In other words, the radius R is the same on an inlet side 6046 of the inflatable body 6016, the outlet side 6048, and all points in between. In addition, the surface of the outer perimeter 6050 of the inflatable body 6016 may be smooth so that the outer perimeter 6050 may be flush with the inner surface of the circumferential wall 6030 when the inflatable body 6016 is fully inflated (e.g., inflated to the maximum extent), thereby closing off at least a portion of the air passage 6034.

Alternatively, the inflatable body 6016 may be constructed so that even when the inflatable body 6016 is inflated to its furthest extent, the gap between the outer perimeter 6050 of the inflatable body 6016 and the inner surface of the circumferential wall 6030 may still exist so that even when the inflatable body 6016 is fully inflated, the air passage 6034 may remain open.

FIG. 6I shows a configuration in which outer perimeter 6050 of the inflatable body 6016 is angled so that the radius R is greater on the outlet side 6048 than on the inlet side 6046 or vice versa. In this configuration, the inflatable body 6016 may be inflated to a maximum extent, about the inner surface of the circumferential wall 6030 and still maintain a gap between the inflatable body 6016 and the circumferential wall 6030 to allow the flow of gas washout through the vent 6000. In this configuration, the cross-sectional width W of the air passage 6034 may be varied from the inlet side 6046 to the outlet side 6048.

Alternatively, the inner surface of the circumferential wall 6030 may be correspondingly angled so that the outer perimeter 6050 of the inflatable body 6016 becomes flush with the inner surface of the circumferential wall 6030 when the inflatable body 6016 is fully inflated. This may create a better seal between the inner surface of the circumferential wall and the outer perimeter 6050 of the inflatable body 6016 while also reducing or minimizing resonance.

FIGS. 6J and 6K respectively illustrate configurations in which a single projection (such as for example, a bead or rim or flange) 6052 or a plurality of projections (such as for example, beads or rims, or flanges) 6052 extend circumferentially in a continuous form around the outer perimeter 6050 of the inflatable body 6016. The projections 6052 may engage the inner surface of the circumferential wall 6030 while leaving one or more gaps between the inflatable body 6016 and the circumferential wall 6030 to allow the gas washout to flow through the air passage 6034 even when the inflatable body 6016 is fully inflated to its maximum allowable extent.

It is contemplated that a similar effect may be achieved by shaping the outer perimeter 6050 to have a curvature in which the radius R of the inflatable body 6016 increases from the inlet and outlet sides 6046, 6048 of inflatable body 6016 toward a midpoint between the inlet and outlet sides 6046, 6048.

It is further contemplated that the outer perimeter 6050 of the inflatable body 6016 may have a plurality of circumferentially aligned projections 6052. In other words, a circumferential path around the outer perimeter 6050 of the inflatable body 6016 may include a plurality of projections 6052. In addition, the projections 6052 may be equally spaced or the spacing between projections 6052 may be unequal. Also, the projections 6052 may have the same circumferential length (i.e., length in the circumferential direction) or may have different circumferential lengths. It should be understood that this intermittent configuration can include one or more rows of projections 6052 (i.e., the configuration illustrated in FIG. 6J or the configuration illustrated in FIG. 6K).

The cap portion 6018 may also include an opening 6054 that is positioned to allow one or more wires 6056 to extend through the housing 6010. The one or more wires 6056 may be attached on one end to the pump 6012 and attached on the other end to the RPT device 4000 to form an electrical connection between the electrical power supply 4210 and/or the central controller 4230 and the pump 6012. Alternatively, the wires 6056 may be connected to the humidifier 5000.

The wires 6056 may convey electrical power to the pump 6012. The wires 6056 may also convey control signals between the pump 6012 and the central controller 4230. The wires 6056 may be routed along the tubes 3350 inside the sleeves 3351 toward the connection port 3600. From there, the wires 6056 may be routed along the air circuit 4170 to the RPT device 4000.

FIG. 6L illustrates another configuration in which the pump 6012 is located remotely to the vent 6000. It should be understood that the components of the vent 6000 illustrated in FIGS. 6A-6K may also be used in the vent 6000 illustrated in FIGS. 6L-6P.

The pump 6012 may be located on the seal-forming structure 3100, in the positioning and stabilising structure 3300, at the humidifier 5000, or at the RPT device 4000. In addition, one or more pneumatic conduits 6058 may be routed along a conduit 6060 of the air circuit 4170. For example, the pneumatic conduits 6058 may be wrapped around the conduit 6060 in a helical formation. FIG. 6M shows the pneumatic conduits 6058 being positioned on the ribs of the conduit 6060. Alternatively, the one or more pneumatic conduits 6058 may be located in the recesses between the ribs.

The flow of the gas in the pneumatic conduits 6058 may be separate from the respiratory gas being delivered to the patient's airways. It is further contemplated that the pressure in the pneumatic conduits 6058 may be less than, the same, or greater than the pressure of the respiratory gas being delivered to the patient's airways and/or the pressure of the gas washout flowing through the vent 6000.

The housing 6010 of the vent 6000 may include an inlet connector 6062 that connects to the one or more pneumatic conduits 6058. The pneumatic conduits 6058 may be secured to the inlet connector 6062 by way of an interference fit, a mechanical fastener, or any other device or method for connecting pneumatic components. The inlet connector 6062 may be connected to the air chamber 6014 so that the pressurized gas conveyed by the one or more pneumatic conduits 6058 flows into the air chamber 6014 and ultimately into the inflatable body 6016. In addition, it is contemplated that even when the pump 6012 is remotely positioned, the housing 6010 may include a pressure relief valve or other pressure relieving device so that the default state of the inflatable body 6016 is deflated or the pressure in the inflatable body 6016 being the same as atmospheric pressure. It is contemplated that the pressure relieving device may be located at the pump. It is further contemplated that the vent 6000 may be located on an elbow for configurations with an elbow connection that connects the seal-forming device 3100 to the air circuit 4170.

### 5.3.5 Connection Port

Connection port 3600 allows for connection to the air circuit 4170.

### 5.3.6 Anti-Asphyxia Valve

In one form, the patient interface 3000 includes an anti-asphyxia valve.

### 5.3.7 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by or formed as part of the chassis 4016.

As shown in FIG. 4C, the RPT device 4000 may have an electrical power supply 4210, one or more input devices 4220, a central controller 4230, a pressure generator 4140, and one or more output devices 4290.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110.

In one form illustrated in FIG. 4B, an inlet air filter 4112 is located at the beginning of the pneumatic path upstream of a pressure generator 4140.

In one form illustrated in FIG. 4B, an outlet air filter 4114, for example an antibacterial filter, is located between an outlet of the pneumatic block 4020 and a patient interface 3000 or 3800.

### 5.4.1.2 Muffler(s)

An RPT device in accordance with one form of the present technology may include a muffler 4120, or a plurality of mufflers 4120.

In one form of the present technology (see e.g., FIG. 4B), an inlet muffler 4122 is located in the pneumatic path upstream of a pressure generator 4140.

In one form of the present technology, an outlet muffler 4124 is located in the pneumatic path between the pressure generator 4140 and a patient interface 3000 or 3800.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH₂O to about 20 cmH₂O, or in other forms up to about 30 cmH₂O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology (see e.g., FIG. 4B), one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000 or 3800.

In one form, a signal from a transducer 4270 may be filtered, such as by low-pass, high-pass or band-pass filtering.

### 5.4.2 RPT Device Electrical Components

### 5.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

As illustrated in FIG. 4C, the power supply 4210 may provide electrical power to the input device 4220, the central controller 4230, the output device 4290, and the pressure generator 4140. The power supply 4210 may also provide electric energy to other components of the RPT device 4000 (or the humidifier 5000, as described above).

### 5.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.2.3 Central Controller

In one form of the present technology, the central controller 4230 is one or a plurality of processors suitable to control an RPT device 4000. The central controller 4230 is show in FIG. 4C.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and/or the humidifier 5000.

The central controller 4230 may be configured to provide output signal(s) to one or more of an output device 4290, a pressure generator 4140, a therapy device controller 4240, a data communication interface 4280, and/or the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.7 Glossary

For the purposes of the present technology disclosure, in certain forms of the present technology, one or more of the following definitions may apply. In other forms of the present technology, alternative definitions may apply.

### 5.7.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar ˜ 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

5.7.1.1 Materials & their Properties

Hardness: Refers to durometer or indentation hardness, which is a material property measured by indentation of an indentor (e.g., as measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.7.1.2 Mechanics

### Axes:

a. Neutral axis: An axis in the cross-section of a beam or plate along which there are no longitudinal stresses or strains.
b. Longitudinal axis: An axis extending along the length of a shape. The axis generally passes through a center of the shape.
c. Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Viscous: The ability of a material to resist flow.

Visco-elasticity: The ability of a material to display both elastic and viscous behaviour in deformation.

Yield: The situation when a material can no longer return back to its original geometry after deformation.

### 5.7.1.3 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

### Thin Structures:

a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

### Thick Structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.7.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.7.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP-EPAP). In some contexts, pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator, or other respiratory therapy device such as an RPT device or portable oxygen concentrator, delivers a volume of breathable gas to a spontaneously breathing patient, it is said to be triggered to do so. Triggering usually takes place at or near the initiation of the respiratory portion of the breathing cycle by the patient's efforts.

### 5.7.4 Anatomy

### 5.7.4.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.7.4.2 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.7.5 Patient Interface

Anti-asphyxia valve (AAV): The component or sub-assembly of a mask system that, by opening to atmosphere in a failsafe manner, reduces the risk of excessive CO₂ rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.7.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.7.6.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.7.6.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical-topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.7.6.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 5.7.6.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.8 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.9 Reference Signs List

| | |
|---|---|
| patient | 1000 |
| bed partner | 1100 |
| patient interface | 3000 |
| nose and mouth patient interface | 3000-1 |
| nose and mouth patient interface | 3000-2 |
| nose-only patient interface | 3000-3 |
| nose-only patient interface | 3000-4 |
| mouth cushion | 3050-1 |
| nose cushion | 3050-2 |
| seal - forming structure | 3100 |
| cushion module | 3150 |
| plenum chamber | 3200 |
| plenum chamber | 3200-1 |
| plenum chamber | 3200-2 |
| chord | 3210 |
| superior point | 3220 |
| inferior point | 3230 |
| plenum chamber inlet port | 3254-1 |
| plenum chamber inlet port | 3254-2 |
| groove | 3266-1 |
| groove | 3266-2 |
| positioning and stabilising structure | 3300 |
| headgear | 3302-1 |
| headgear | 3302-2 |
| inferior strap | 3304-1 |
| superior strap | 3305-1 |
| magnetic member | 3306-1 |
| rear strap | 3307-1 |
| rear strap | 3307-2 |
| intermediate section | 3308-2 |
| slit | 3309-2 |
| strap | 3310 |
| tab | 3320 |
| concertina structure | 3328 |
| concertina structure | 3328-1 |
| inlet | 3332 |
| Inlet | 3332-1 |
| rigidiser arm | 3340 |
| first end | 3342 |
| second end | 3344 |
| tube | 3350 |
| sleeve | 3351 |
| superior opening | 3352 |
| inferior extension | 3354 |
| connection member | 3356 |
| non - extendable tube sections | 3363 |
| magnet | 3370-1 |
| four - point arm sleeve | 3380 |
| conduit sleeve | 3380-1 |
| inferior extension | 3384 |
| connection member | 3386 |
| inferior opening | 3388-1 |
| tabs | 3394 |
| tabs | 3394-1 |
| vent | 3400 |
| vent opening | 3402-1 |
| vent opening | 3402-2 |
| conduit connection structure | 3500 |
| arm connection structure | 3504 |
| connection port | 3600 |
| elbow | 3610 |
| forehead support | 3700 |
| RPT device | 4000 |
| external housing | 4010 |
| upper portion | 4012 |
| portion | 4014 |
| panel | 4015 |
| chassis | 4016 |
| handle | 4018 |
| pneumatic block | 4020 |
| air filter | 4110 |
| inlet air filter | 4112 |
| outlet air filter | 4114 |
| muffler | 4120 |
| inlet muffler | 4122 |
| outlet muffler | 4124 |
| pressure generator | 4140 |
| blower | 4142 |
| motor | 4144 |
| anti - spill back valve | 4160 |
| air circuit | 4170 |
| supplementary gas | 4180 |
| electrical components | 4200 |
| single Printed Circuit Board Assembly PCBA | 4202 |
| power supply | 4210 |
| input device | 4220 |
| central controller | 4230 |
| humidifier | 5000 |
| humidifier inlet | 5002 |
| humidifier outlet | 5004 |
| humidifier base | 5006 |
| reservoir | 5110 |
| conductive portion | 5120 |
| humidifier reservoir dock | 5130 |
| locking lever | 5135 |
| vent | 6000 |
| housing | 6010 |
| pump | 6012 |
| air chamber | 6014 |
| inflatable body | 6016 |
| cap portion | 6018 |
| base portion | 6020 |
| outlet opening | 6022 |
| flange | 6024 |
| flange | 6026 |
| channel | 6028 |
| circumferential wall | 6030 |
| inlet opening | 6032 |
| air passage | 6034 |
| flow of gas washout | 6036 |
| chamber | 6038 |
| inlet | 6040 |
| outlet | 6042 |
| air passage | 6044 |
| inlet side | 6046 |
| outlet side | 6048 |
| outer perimeter | 6050 |
| projection | 6052 |
| wires | 6054 |
| pneumatic conduits | 6058 |
| conduit | 6060 |
| inlet connector | 6062 |

1. A pneumatically controlled gas washout vent configured to vent respiratory gas from a patient interface, the gas washout vent comprising:
an inflatable body;
a pump configured to pressurize the inflatable body;
housing that enclosed the inflatable body and the pump; and
an airflow passage extending between the inflatable body and a wall of the housing,
wherein the inflatable body is configured to expand upon being inflated by the pump and narrow the airflow passage upon being inflated.

2. The gas washout vent of claim 1, wherein a central region of the vent is pneumatically sealed.

3. The gas washout vent of claim 2, wherein the housing comprises a cap portion positioned on an exterior of the patient interface, and a base portion positioned inside the patient interface.

4. The gas washout vent of claim 3, wherein the cap portion comprises one or more outlet openings, and wherein the airflow passage is configured to discharge gas washout through the one or more outlet openings.

5. The gas washout vent of claim 4, wherein one or more inlet openings are positioned around a perimeter of the base portion.

6. The gas washout vent of claim 5, wherein the inflatable body is secured to the base portion.

7. The gas washout vent of claim 6, further comprising an air chamber configured to receive a pneumatic output of the pump and configured to discharge gas to the inflatable body.

8. The gas washout vent of claim 7, wherein the inflatable body is configured to be in a deflated state when the pump is disabled.

9. The gas washout vent claim 8, wherein the inflatable body is configured so that when the inflatable body is deflated, a cross-sectional width of the airflow passage is greatest.

10. The gas washout vent of claim 9, wherein the inflatable body is configured to be incrementally inflated and deflated.

11. The gas washout vent of claim 9, wherein the inflatable body is configured to be dynamically inflated and deflated.

12. The gas washout vent of claim 11, wherein an outer perimeter of the inflatable body is inclined so that a radius of the inflatable body on one side is greater than a radius of the inflatable body on the other side.

13. The gas washout vent of claim 11, wherein an outer perimeter of the inflatable body has a circumferential bead or flange or intermittent beads or flanges.

14. The gas washout vent of claim 13, wherein the pump is one of a diaphragm pump, a piezoelectric pump, or a peristaltic pump.

15. A patient interface assembly comprising:
a sealing structure;
a positioning and stabilizing structure configured to support the sealing structure; and
the gas washout vent of claim 14.

16. A pneumatically controlled gas washout vent configured to vent respiratory gas from a patient interface, the gas washout vent comprising:
an inflatable body;
a pneumatic inlet configured to receive pressurized gas;
an air chamber configured to receive the pressurized gas from the pneumatic inlet and discharge the pressurized gas to the inflatable body;
housing that enclosed the inflatable body; and
an airflow passage extending between the inflatable body and a wall of the housing,
wherein the inflatable body is configured to expand upon being inflated by the pressurized gas and narrow the airflow passage upon being inflated.

17. The gas washout vent of claim 16, wherein a central region of the vent is pneumatically sealed.

18. The gas washout vent of claim 17, wherein the housing comprises a cap portion positioned on an exterior of the patient interface, and a base portion positioned inside the patient interface.

19. The gas washout vent of claim 18, wherein the cap portion comprises one or more outlet openings, and wherein the airflow passage is configured to discharge gas washout through the one or more outlet openings.

20. The gas washout vent of claim 19, wherein one or more inlet openings are positioned around a perimeter of the base portion.

21. The gas washout vent of claim 20, wherein the inflatable body is secured to the base portion.

22. The gas washout vent of claim 21, wherein the inflatable body is configured to be in a deflated state when a flow of the pressurized gas to the pneumatic inlet is terminated.

23. The gas washout vent of claim 22, wherein the inflatable body is configured so that when the inflatable body is deflated, a cross-sectional width of the airflow passage is greatest.

24. The gas washout vent of claim 23, wherein the inflatable body is configured to be incrementally inflated and deflated.

25. The gas washout vent of claim 24, wherein the inflatable body is configured to be dynamically inflated and deflated.

26. The gas washout vent of claim 25, wherein an outer perimeter of the inflatable body is inclined so that a radius of the inflatable body on one side is greater than a radius of the inflatable body on the other side.

27. The gas washout vent of claim 25, wherein an outer perimeter of the inflatable body has a circumferential bead or flange or intermittent beads or flanges.

28. A patient interface assembly comprising:
a sealing structure;
a positioning and stabilizing structure configured to support the sealing structure; and
the gas washout vent of claim 1.

29. An RPT system comprising:
the patient interface assembly of claim 28;
an air delivery conduit configured to supply a first flow of pressurized gas to the patient interface assembly; and
a pump configured to supply a second flow of pressurized gas to the gas washout vent.

30. The RPT system of claim 29, wherein the pump is one of a diaphragm pump, a piezoelectric pump, or a peristaltic pump.
